SEQUENCE LISTING (4 sequences)

SEQ ID NO: 1 - protein, 60 aa, Sus scrofa
LQNEVFVVPNHMLLSVNFGPTVDGDFLTDLPDTLLQLGQFKKTQILVGVNKDEGTAFLVY

SEQ ID NO: 2 - protein, 4 aa, Homo sapiens
YGTP

SEQ ID NO: 3 - protein, 4 aa, Sus scrofa
NHML

SEQ ID NO: 4 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

(19) United States
(12) Patent Application Publication
(10) Pub. No.: US 2021/0277368 A1
(43) Pub. Date: Sep. 9, 2021

(54) COMPOSITIONS AND METHODS FOR TREATMENT OF CHEMICAL WARFARE AGENTS

(71) Applicants: Battelle Memorial Institute, Columbus, OH (US); The Ohio State University, Columbus, OH (US)

(72) Inventors: Kevin G. McGarry, JR., Delaware, OH (US); Robert A. Moyer, Plain City, OH (US); David W. Wood, Dublin, OH (US)

(21) Appl. No.: 16/783,383

(22) Filed: Feb. 6, 2020

Related U.S. Application Data
(60) Provisional application No. 62/803,006, filed on Feb. 8, 2019.
(60) Provisional application No. 62/809,900, filed on Feb. 25, 2019.
(60) Provisional application No. 62/935,694, filed on Nov. 15, 2019.

(51) Int. Cl. C12N 9/18; A61M 5/20

(52) U.S. Cl. CPC C12N 9/18; C12Y 301/01008; A61M 5/20

(57) ABSTRACT

Disclosed herein are proteins having at least 90% sequence identity to a wild-type human butyrylcholinesterase and compositions comprising same. The disclosed proteins may have at least one mutation at a position within the acyl binding pocket and at least one mutation adjacent to the acyl biding pocket. Further disclosed are proteins having at least 90% sequence identity to a wild-type human butyrylcholinesterase, wherein the protein may comprise a mutation at a position selected from one or more of 282, 283, and 284.

### CROSS-REFERENCE TO RELATED APPLICATIONS

This application claims priority to and benefit of U.S. Ser. No. 62/803,006, filed on Feb. 8, 2019, U.S. Ser. No. 62/809,900 filed Feb. 25, 2019, and U.S. Ser. No. 62/935,694 filed Nov. 15, 2019, the contents of each are incorporated herein in their entirety.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 16783383_SequenceListing.txt, the date of creation of the ASCII text file is Jun. 1, 2020, and the size of the ASCII text file is 10 KB.

### BACKGROUND

Organophosphorus (OP) cholinesterase inhibitors, including chemical warfare nerve agents (CWNAs) such as sarin and VX, continue to be a global threat against both military personnel and civilian populations. From conflict zones in third world nations to terrorist attacks in the western world, CWNAs may be employed in a multitude of different ways to wreak havoc on society (FDA, 2018). Inhibition of acetylcholinesterase (AChE) via phosphylation of the active serine site (Marrs, 1993) remains the primary mechanism of toxicity through which CWNAs act. The resulting inhibition results in an accumulation of acetylcholine thereby creating a cholinergic crisis that drives respiratory failure (Giyanwani et al., 2017) which can ultimately be fatal. CWNAs also inhibit butyrylcholinesterase (BChE) without any apparent toxic effects; rather, BChE acts as a bioscavenger that binds circulating CWNAs and removes them from circulation (Golomb, 2008). BChE retains a high amount of structural similarity to AChE (Vellom et al., 1993), and like AChE can also hydrolyze the neurotransmitter acetylcholine. Early animal studies using BChE as a bioscavenging treatment strategy for CWNA poisoning showed long-term resistance to poisoning after intravenous or intramuscular injections of BChE from isolated human serum (Lange et al., 2001). The current standard-of-care for CWNA poisoning includes a three-fold approach, treating those afflicted with muscarinic antagonists (e.g. atropine), AChE reactivators (oximes) (Seidler et al., 1996), and an anticonvulsant (e.g. diazepam) as needed. Despite efforts spanning decades and the creation of numerous oxime compounds, the standard therapy for CWNA poisoning relies on chemicals developed over fifty years ago (Yanagisawa et al., 2006). Additionally, the effectiveness of oxime therapy to treat CWNA poisoning strongly depends on the specific CWNA used as well as the circumstances surrounding exposure. Even so, the dual combination of an atropine-oxime aid via auto-injectors remains the accepted therapy for both civilian and military personnel (Blouin et al., 2016).

Accordingly, improved compositions and methods for the treatment of OP poisoning are needed. The instant disclosure seeks to address this need in the art.

### BRIEF SUMMARY

Disclosed herein are proteins having at least 90% sequence identity to a wild-type human butyrylcholinesterase and compositions comprising same. The disclosed proteins may have at least one mutation at a position within the acyl binding pocket and at least one mutation adjacent to the acyl biding pocket. Further disclosed are proteins having at least 90% sequence identity to a wild-type human butyrylcholinesterase, wherein the protein may comprise a mutation at a position selected from one or more of 282, 283, and 284.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Whole blood was obtained from 10 male and 10 female animals per species and processed to red blood cell membranes and plasma as described. Panel A displays AChE activity from the RBC membrane preparations of each individual using acetylthiocholine iodide as the substrate. Panel B displays BChE activity from the plasma preparations of each individual using butyrylthiocholine iodide as the substrate. Each point represents the mean activity from a single sample measured in triplicate in two independent experiments. A two-way Anova with a Tukey's multiple comparisons test was utilized to assess statistical significance. P<0.05-0.01 (*), P<0.001-0.01 (**), P<0.001-0.0001 (***) P<0.0001 (****).

FIG. 2. Aging and/or spontaneous reactivation of sarin-inhibited cholinesterases. Activity of sarin-inhibited AChE or BChE in the presence or absence of 2-PAM Cl. Sarin-inhibited RBC membranes from human (A), Yorkshire swine (B) or sarin-inhibited plasma preparations from human (C), Yorkshire swine (D) were incubated at 37° C. until 2-PAM was added and the enzyme activity measured at the times indicated. Data represent average±SEM performed in triplicate. Aging half times were calculated using a non-linear regression model. For simplicity, only the sarin results are displayed above as aging was not observed with VX. Further, only human and one swine model are displayed as all of the primates evaluated yielded similar results and the Gottingen mini pig displayed results similar to the Yorkshire swine. The calculated spontaneous reactivation of plasma BChE following a sarin challenge was 5.0 hours (6.7 hours for the Gottingen mini pig).

FIG. 3. Reactivation of VX-inhibited AChE and BChE by 2-PAM Cl. Oxime reactivation of VX-inhibited AChE and BChE was performed as described. Background due to oximolysis was accounted for in each calculation.

FIG. 4. Sequence alignment comparison of human BChE vs. porcine (Sus scrofa) BChE and sequence of wild type Human BChE Mature Protein Sequence (bottom panel). The red letters indicate differences between the two species. The highlighted region represents the acyl binding pocket of the enzyme. In the bottom panel, amino acid residues 282-285 have been highlighted to identify the location of the mutations of the hybrid enzyme. In the hybrid enzyme described herein, the native YGTP (SEQ ID NO: 2) sequence has been changed to NHML (SEQ ID NO: 4).

FIG. 5. Anti-Histidine Western blot following the expression and purification of indicated BChE constructs.

FIG. 6. Silver stain following the expression and purification of human WT BChE (left) and human porcinated BChE (right).

FIG. 7. Substrate titration comparison of WT BChE vs. porcinated human BChE

FIG. 8. Inhibition of multiple recombinant BChEs by sarin. As a reference, the estimated IV LD50 and the Kd of the G117H BChE variant are included. Each point represents mean±SEM of triplicate measurements.

FIG. 9. Catalytic degradation of sarin by porcinated BChE. Panel A) Percent activity/reactivation of both wild-type and porcinated BChE measured in presence or absence of the oxime reactivator, 2 PAM-Cl. The enzymes were subjected to the aging experiments as described previously. Following the removal of unbound agent using spin columns, reactions were incubated from 0 to 22 hours at 37° C. prior to the addition of 2 PAM-Cl and activity was determined using the Ellman's method as described. Error represents±the standard error of the mean. Panel B) Concentration of IMPA, ng/mL, measured by LC-MS/MS analysis at 0, 1, 2, 4, 6, and 22 hours using aliquots from inhibition reaction described in (A). The concentration of IMPA that was detected in a sarin control (i.e. in MOPS buffer alone) is plotted at the various time points on the right y-axis to show the degradation sarin in the absence of enzyme.

FIG. 10. Extracted Ion Chromatograms for Sarin and IMPA. Standard sarin (retention time=3.66 min) and IMPA (retention time=1.56 min) controls are displayed in panel A showing the experimentally observed peaks. These peaks are displayed at the expected retention times for these two compounds based off previous experience with these analytes using this LC-MS/MS method. Panel B displays a zoomed in capture of the IMPA chromatogram at each time point from 0 to 22 hours post challenge. As can clearly be seen, the abundance of IMPA increases over time, corresponding to the reactivation of the inhibited enzyme.

FIG. 11. Proposed pathways for spontaneous hydrolysis by conserved water molecules with sarin-inhibited BChE

FIG. 12. Cluster analysis by RMSD for the native forms of BChE color coded by relative populations of each cluster where blue is most populated, red is 2nd most populated, green is 3rd most populated, yellow is 4th most populated, pink is 5th most populated, and black is any cluster more than 5th. Root mean square fluctuation analysis for the native enzyme: The root mean square fluctuation (RMSF) was calculated using AMBER's CPPTRAJ module for each residue in order to evaluate the flexibility of certain residues for the native enzyme. In order to compare the flexibility of the mutation site to the rest of the protein it is color-coded.

FIG. 13. RMSF analysis for the native forms of BChE where the site of the mutation is color-coded red.

FIG. 14. Active site distances native forms of BChE color-coded by the different interactions in the active site. Distances are displayed as heat maps and are shown as a function of color over time.

FIG. 15. Distances of the acyl loop and the site of the mutation for the native forms of BChE to evaluate different interactions near the mutation. Distances are displayed as heat maps and are shown as a function of color over time.

FIG. 16. Distances and angle of Trp82 in the Ω-loop as compared to the position and orientation of the ε-nitrogen of the catalytic His438 located in the active site.

FIG. 17. Illustration of “acyl-pocket encroachment” in the HID form of the native enzyme.

FIG. 18. Illustration of active site “climbing” in the HIE form of the native enzyme.

FIG. 19. Molecular Dynamics analysis of the inhibited enzyme before and after mutation. Moleculer dynamics (MD) simulations were carried out for WT and hybrid forms of inhibited BChE, that included each possible protonation states for the His283 in the hybrid form of the enzyme. All MD simulations were performed with the AMBER 16 molecular dynamics package with the ff03 force field. The protein preparation was performed for the WT and hybrid forms of the enzyme with 2XQJ and 2XQK that are the (R)- and (S)-inhibited stereoisomers of VX, respectively, as previously eluded to in the computational methods. Clustering analysis for the inhibited enzyme: A clustering protocol using AMBER's CPPTRAJ module was used in order to decrease the amount of structures for analysis in the inhibited forms of the enzyme. A “representative” structure was chosen for each cluster that is color-coded by the relative populations of each cluster. Cluster analysis by RMSD for the 2XQJ inhibited forms of BChE color coded by relative populations of each cluster where blue is most populated, red is 2^{nd} most populated, green is 3^{rd} most populated, yellow is 4^{th} most populated, pink is 5^{th} most populated, and black is any cluster more than 5^{th}.

FIG. 20. Cluster analysis by RMSD for the 2XQK inhibited forms of BChE color coded by relative populations of each cluster where blue is most populated, red is 2nd most populated, green is 3rd most populated, yellow is 4th most populated, pink is 5th most populated, and black is any cluster more than 5th. Root mean square fluctuation analysis for the inhibited enzyme: The root mean square fluctuation (RMSF) was calculated using AMBER's CPPTRAJ module for each residue in order to evaluate the flexibility of certain residues for the inhibited enzyme. In order to compare the flexibility of the mutation site to the rest of the protein it is color-coded.

FIG. 21. RMSF analysis for the 2XQJ inhibited forms of BChE where the site of the mutation is color-coded red.

FIG. 22. RMSF analysis for the 2XQK inhibited forms of BChE where the site of the mutation is color-coded red.

FIG. 23. Active site distances for the 2XQJ inhibited forms of BChE color-coded by the different interactions in the active site. Distances are displayed as heat maps and are shown as a function of color over time.

FIG. 24. Active site distances for the 2XQK inhibited forms of BChE color-coded by the different interactions in the active site. Distances are displayed as heat maps and are shown as a function of color over time.

FIG. 25. Distances of the acyl loop and the site of the mutation for the 2XQJ inhibited forms of BChE to evaluate different interactions near the mutation. Distances are displayed as heat maps and are shown as a function of color over time.

FIG. 26. Distances of the acyl loop and the site of the mutation for the 2XQK inhibited forms of BChE to evaluate different interactions near the mutation. Distances are displayed as heat maps and are shown as a function of color over time.

FIG. 27. Distances and angle of Trp82 in the Ω-loop as compared to the position and orientation of the ε-nitrogen of the catalytic His438 located in the active site.

FIG. 28. Distances and angle of Trp82 in the Ω-loop as compared to the position and orientation of the ε-nitrogen of the catalytic His438 located in the active site.

FIG. 29. Water interaction distances for the 2XQJ inhibited forms of BChE color-coded by the different interactions in the active site. Distances are displayed as heat maps and are shown as a function of color over time.

FIG. 30. Water interaction distances for the 2XQK inhibited forms of BChE color-coded by the different interactions in the active site. Distances are displayed as heat maps and are shown as a function of color over time.

FIG. 31. Example of an “active” water for Pathway 2 that sits in between the phosphylated serine (SIB) and Glu197. This is taken from the most populated cluster of the HIE form of 2XQK inhibition.

FIG. 32. Example of an “active” water for Pathways 1 and 2 that sits in between the phosphylated serine (SIB) and Glu197. This is taken from the third most populated cluster of the HIE form of 2XQK.

FIG. 33. Illustration of the water pocket formed in between Glu197 and phosphylated Ser198 (Sarin198) for the top 3 clusters of the HIE form of 2XQK. Each of the individual clusters is color coded by population: blue, most populated; red, next most populated; and green, lowest population.

FIG. 34. % BchE Activity vs Time in hours for recombinant human WT BChE vs GB (top) and recombinant human-pig acyl BChE vs GB (bottom).

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Sequence identity” as used herein indicates a nucleic acid sequence or amino acid sequence that has the same nucleic acid sequence or amino acid sequence as a reference sequence or has a specified percentage of nucleotides or amino acids that are the same at the corresponding location within a reference sequence when the two sequences are optimally aligned. For example, a nucleic acid sequence or amino acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides or amino acids, or the full-length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Chemical warfare nerve agents (CWNAs) such as sarin and VX are some of the most potent neurotoxicants known. CWNAs belong to a class of chemicals known as organophosphates (OPs). Many pesticides also belong to this class of chemicals; however, they are generally much less potent than CWNAs. It is widely understood that the primary mechanism of OP toxicity is due to inhibition of acetylcholinesterase (AChE) via phosphylation of the active serine site (Chambers 1992; Eto 1974; Marrs 1993; Wilson et al. 1992). It is this phosphylation event that can have devastating consequences to a poisoned individual.

In cellular signaling involving cholinergic neurons, the neurotransmitter acetylcholine (ACh) is packaged and released into the synaptic cleft where it binds to and activates ACh-receptors, continuing a downstream signaling event. To attenuate the signaling event, AChE hydrolyzes excess ACh, thereby terminating neuronal signaling. As such, AChE is an essential enzyme in the regulation of neuronal cellular signaling. Inhibition of AChE results in the accumulation of ACh, leading to continual stimulation of ACh receptors and may ultimately lead to cholinergic crisis. During cholinergic crisis, the continuous stimulation of cholinergic neurons and/or skeletal muscles may result in seizures, paralysis, and ultimately respiratory failure and death (Eddleston et al. 2008). The current FDA-approved/fielded standard of care for OP poisoning includes the use of three drugs: 1) A muscarinic antagonist (e.g., atropine); 2) An AChE reactivator (e.g., pralidoxome); 3) A benzodiazepine (e.g. diazepam), to control seizures.

Butyrylcholinesterase (BChE) exhibits a high degree of structural similarity and sequence homology to AChE (Vellom et al. 1993), and, like AChE, can hydrolyze the neurotransmitter acetylcholine and is also inhibited by CWNAs. BChE is believed to have arisen early in the evolution of vertebrates due to a gene duplication event and the structural similarities of the two enzymes are striking (Soreq and Zakut 1993). Most notably, the catalytic triad of BChE, like AChE, resides at the bottom of a 20 Å deep gorge that is lined with aromatic amino acid residues. However, while AChE has fourteen aromatic residues lining its gorge which facilitates pi-pi stacking interactions, BChE retains only eight (Nicolet et al. 2003). Interestingly, the endogenous function of BChE remains largely unknown as individuals deficient in active BChE appear to lead normal, healthy lives (Manoharan et al. 2007). BChE, however, is thought to serve in a number of biological processes, namely, neuronal cellular development, neuronal signaling, and it appears to have a role in bronchial airway smooth muscle function (Lockridge 2015; Masson and Lockridge 2010).

One significant function of BChE, aside from the redundancy in function with AChE, is BChE's ability to serve as a bioscavenger of toxic esters. It is the difference in the volume of the gorge of the two enzymes discussed above that allows BChE to hydrolyze a wider variety of substrates and thus serve in this capacity (Darvesh et al. 2003).

The concept of using exogenous BChE as a bioscavenging treatment for CWNA poisoning has been evaluated in animal studies (Lockridge 2015). The results of multiple studies have demonstrated that exogenous BChE is protective against OP poisoning, although large amounts of BChE are required. For example, more than 20 mg/kg of BChE was required to protect guinea pigs against a 2.5× LD50 percutaneous challenge of the nerve agent, VX (Mumford et al. 2013; Mumford et al. 2011). These results are promising and indeed BChE is being evaluated as a medical countermeasure for CWNA poisoning in early clinical trials. However, the binding of CWNAs to BChE is stoichiometric and irreversible (i.e. one molecule of BChE is required to remove one molecule of CWNA). As such, the amount of BChE that must be administered must match the level of CWNA, imposing a limit to the OP challenge level that can be protected against due to the limitations on how much exogenous protein may be administered safely. Furthermore, the volume of human serum required for purification and the cost associated with the purification effort pose significant challenges to fielding BChE as a medical countermeasure (Lockridge 2015; Lushchekina et al. 2018; Nachon et al. 2013; Rice et al. 2016; Saxena et al. 2008).

The mutagenization of BChE to create a catalytic or self-reactivating bioscavenger has been extensively explored and reviewed (Lushchekina et al. 2018). In brief, Lockridge and colleagues discovered that a point mutation (G117H) within the oxyanion hole of the enzyme confers organophosphate hydrolase (OPH) characteristics to BChE (Lockridge et al. 1997). This discovery has led to two decades of research regarding the creation and optimization of catalytic and pseudocatalytic (oxime-assisted) bioscavengers based on wild type human BChE. Relatively recent developments involving several species and various mutations indicates that increased flexibility within the acyl loop of both pig BChE and bovinated-human BChE appears to promote auto-reactivation following inhibition by VX or chloropyrifos oxon (Brazzolotto et al. 2015; Dafferner et al. 2017; Dorandeu et al. 2008; Terekhov et al. 2017). To date, however, neither the G117H mutation alone, nor in combination with other mutations, has yielded an enzyme that displays sufficient activity required for a catalytic bioscavenger (i.e. K_{cat}/K_{M}>10⁵ M⁻¹s^{-l}; (Vachon et al. 2013)) while retaining sufficient binding affinity to surpass the effectiveness of wildtype BChE as a medical countermeasure for OP poisoning.

Disclosed herein are novel human-porcine hybrid BChEs that may be used to catalytically degrade the CWNA sarin while retaining near wild-type binding affinity.

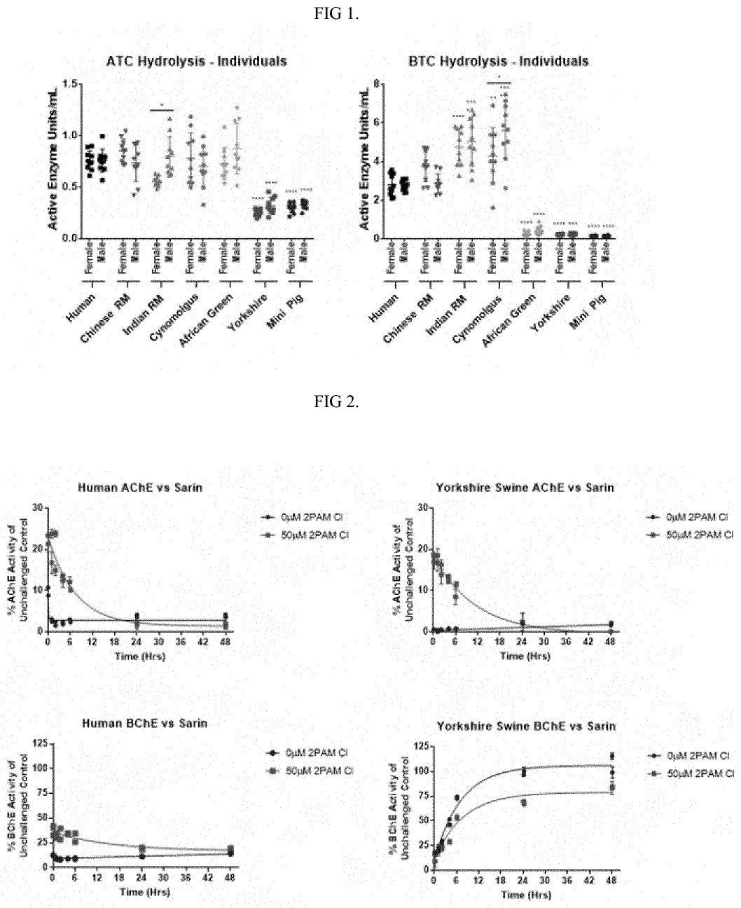

In one aspect, a composition comprising a modified human butyrylcholinesterase having at least 90%, 91%, 92%, 93%, 94%, 95%, 96%, 97%, 98%, or 99%, sequence identity to wild-type human butyrylcholinesterase as described in UniProtKB/Swiss-Prot: P06276.1 is disclosed. A variant in accordance with the disclosed invention is shown in FIG. 1, panel B. The protein may comprise at least one, or at least two, or at least three mutations at a position within the wild-type human butyrylcholinesterase acyl binding pocket at amino acids 284-288, and at least one, or at least two, or at least three mutations adjacent to the wild-type human butyrylcholinesterase acyl biding pocket. By adjacent, it is meant either downstream or upstream of the binding pocket sequence, or, in other aspects, physically close to the binding pocket when folded three dimensionally.

In other aspects, the modified human butyrylcholinesterase may one or more mutations adjacent to a butyrylcholinesterase binding site, and one or more mutations within said butyrylcholinesterase binding site, wherein the mutation increases flexibility of the butyrylcholinesterase protein as compared to wild type human butyrylcholinesterase. In another aspect, the mutation may be one which increases the catalytic capacity of BChE towards the degradation of cholinesterase inhibitors.

In one aspect, the modified human butyrylcholinesterase may have at least 90% sequence identity to wild-type human butyrylcholinesterase, and may comprise a mutation at a position selected from 282, 283, 284, 285, and combinations thereof.

In one aspect, the modified human butyrylcholinesterase may comprise a mutation at position 282 and 283. In one aspect, the protein may comprise a mutation at position 282, 283, and 284. In one aspect, the protein may comprise a mutation at position 282, 283, 284, and 285. It will be understood to one of ordinary skill in the art that the protein may comprise further mutations that do not affect the activity and/or folding of the protein, such mutations being silent mutations and within the scope of the invention.

In one aspect, the modified human butyrylcholinesterase may comprises at least one mutation, or at least two mutations, or at least three mutations selected from Y282N, G283H, and T284M and combinations thereof, and may further include the mutation, P285L.

In one aspect, the modified human butyrylcholinesterase may comprise a Y282N mutation, a G283H mutation, a T284M mutation, and a P285L mutation.

In one aspect, the modified human butyrylcholinesterase protein may have a mutation at position 282, 283, 284, and 285 with reference to human wild-type butyrylcholinesterase. The mutation may be one, two, or three mutations selected from Y282N, G283H, and T284M. Optionally, P285L may additionally be mutated. In one aspect, the protein may comprise all three of Y282N, G283H, and T284M. In one aspect, the mutation may comprise all four Y282N, G283H, and T284M, and P285L.

In one aspect, a method of treating exposure to organophosphate exposure in an individual in need thereof is disclosed. In this aspect, the method comprises the step of administering a composition comprising a modified human butyrylcholinesterase as described herein, to an individual in need thereof. Such administration may occur before, during or after CWNA (organophosphate) exposure. Dosing may occur at predetermined intervals, such as twice a day, daily, weekly, or other similar intervals, the determination of such administration being within the skill in the art, taking into account the individual and the expected or determined level of exposure to the CWNA.

Pharmaceutical Compositions

In one aspect, the modified human butyrylcholinesterase protein may be altered by adding inert structural components to the structure of the protein in order to enhance storage stability and/or circulatory half-life. Examples include but are not limited to polyethylene glycol (PEGylation) and glycan (glycosylation). Furthermore, the modified human butyrylcholinesterase protein may be altered and/or co-administered in conjunction with another therapeutic strategy in order to enhance its blood brain barrier permeability. Examples include, but are not limited to, nanoparticle encapsulation, focused ultrasound, and viral vectors.

In one aspect, a composition comprising one or more of the aforementioned mutated butyrylcholinesterase proteins as a first active agent, and a pharmaceutically acceptable carrier or excipient, is disclosed. In one aspect, the composition my further comprise a second active agent, wherein the second agent may be an anti CWNA agent. For example, in one aspect, the anti-CWNA may be selected from one or more of atropine, and 2-PAM. The composition may comprise a first active agent that is a variant of butyrylcholinesterase protein as described above, in combination with a second active agent, wherein the second active agent is an anti-CWNA agent and may be enclosed in a device capable of auto-injection into an individual in need thereof. For example, the one or more of the aforementioned mutated butyrylcholinesterase proteins may be included in an atropine autoinjector, wherein 2 mg of atropine is provided in 0.7 cc, or in a pralidoxime autoinjector (2-PAM), 600 mg wherein the pralidoxime is provided in in 2 cc. Alternatively, the one or more of the aforementioned mutated butyrylcholinesterase proteins may be included in an autoinjector having both atropine and 2-PAM.

In one aspect, active agents provided herein may be administered in a dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating the conditions described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1. Characterization of Cholinesterases from Multiple Large Animal Species for Medical Countermeasure Development

Organophosphorus (OP) compounds, which include insecticides and chemical warfare nerve agents (CWNAs) such as sarin (GB) and VX, continue to be a global threat to both civilian and military populations. It is widely accepted that cholinesterase inhibition is the primary mechanism for acute OP toxicity. Disruption of cholinergic function through the inhibition of acetylcholinesterase (AChE) leads to the accumulation of the neurotransmitter acetylcholine. Excess acetylcholine at the synapse results in an overstimulation of cholinergic neurons which manifests in the common signs and symptoms of OP intoxication (miosis, increased secretions, seizures, convulsions, respiratory failure). The primary therapeutic strategy employed in the U.S. to treat OP intoxication includes reactivation of inhibited AChE with the oxime pralidoxime (2-PAM) along with the muscarinic acetylcholine receptor antagonist atropine and the benzodiazepine diazepam. CWNAs are also known to inhibit butyrylcholinesterase (BChE) without any apparent toxic effects. Therefore, BChE may be viewed as a “bioscavenger” that stoichiometrically binds CWNAs and removes them from circulation. The degree of inhibition of AChE and BChE and the effectiveness of 2-PAM are known to vary between species. Animal models are imperative for evaluating the efficacy of CWNA medical countermeasures, and a thorough characterization of available animal models is important for translating results to humans. Thus, the objective of this study was to compare the circulating levels of each of the cholinesterases as well as multiple kinetic properties (inhibition, reactivation, and aging rates) of both AChE and BChE derived from humans to AChE and BChE derived from commonly used large animal models.

Organophosphorus (OP) cholinesterase inhibitors, including chemical warfare nerve agents (CWNAs) such as sarin and VX, continue to be a global threat against both military personnel and civilian populations. Inhibition of the enzyme acetylcholinesterase (AChE) via phosphylation of the active serine site is the primary mechanism of toxicity of CWNAs. Inhibition of AChE prevents it from carrying out its normal physiological function (i.e., hydrolysis of the neurotransmitter acetylcholine). The resulting accumulation of acetylcholine leads to hyperstimulation of acetylcholine receptors at the neuromuscular junction and cholinergic neuronal synapses, which can trigger a cholinergic crisis that may ultimately result in respiratory failure and death (Eddleston et al., 2008). Pharmacotherapy for OP-induced cholinergic effects typically includes the muscarinic antagonist atropine along with an oxime reactivator, the latter of which removes the OP from the enzyme (Cannard, 2006; Eyer, 2003; Thiermann et al., 2013). If needed, a benzodiazepine (e.g. diazepam) may also be administered to control seizures.

OPs (including CWNAs) also bind to and inhibit butyrylcholinesterase (BChE), a related enzyme that is sequentially and structurally similar to AChE [51-54% sequence identity in mammalian species; (Vellom et al., 1993)] that can also hydrolyze acetylcholine. The normal physiological function of BChE is not clear and it is not essential for life, as evidenced by the existence of genetic variations that result in a lack of BChE activity in apparently healthy people (Manoharan et al., 2007). Further evidence for the lack of a critical function of BChE may be found in mice, where genetic ablation of the BCHE gene yields healthy animals with no obvious phenotype (Li et al., 2008). Therefore, it is not surprising that inhibition of BChE does not cause toxicity in humans or laboratory animals, and BChE's role in the biological response to OP poisoning is primarily that of a bioscavenger that stoichiometrically binds the compounds with high affinity, effectively removing them from circulation. BChE is a soluble protein that is found in circulation and this trait, along with its capacity for binding CWNAs, has led to the ongoing development of exogenous BChE as a medical countermeasure (MCM) for OP poisoning (Reed et al., 2017).

Though effective, the currently approved MCMs for CWNA poisoning have significant limitations. For instance, the poor blood brain barrier permeability of most oximes restricts their capacity for reactivating inhibited AChE in the CNS, and the efficacy of individual oximes is highly dependent on the identity of the OP and the time elapsed between exposure and treatment (Lorke et al., 2008). Efforts to develop improved MCMs are complicated by interspecies variability as well as other factors; the efficacy of individual oximes has been found to vary considerably depending on the animal species, experimental model, and specific OP tested (Worek et al., 2016). Efforts to develop improved MCMs are ongoing in laboratories across the world, and selection of appropriate animal models is one of the major challenges faced by researchers. In the United States, the Food and Drug Administration's Animal Rule permits the approval of MCMs for use in humans based on the results of animal efficacy studies when human efficacy studies would not be ethical and/or feasible (e.g., CWNA poisoning, US Food and Drug Administration, Product development under the Animal Rule: Guidance for industry; 2015. http://www.fda.gov/Drugs/GuidanceComplianceRegulatoryInformation/Guidances/ucm3992 00.htm and Snoy, P. J., Establishing efficacy of human products using animals: the US food and drug administration's “animal rule”. Vet Pathol, 2010. 47(5): p. 774-8.) (Snoy, 2010). It is generally recommended that MCM efficacy be demonstrated in more than one species that is expected to react with a response predictive of the human response (US Food and Drug Administration, Product development under the Animal Rule: Guidance for industry; 2015. http://www.fda.gov/Drugs/GuidanceComplianceRegulatoryInformation/Guidances/ucm3992 00.htm). Due to their close evolutionary relationship and similarities to humans, nonhuman primates (esp. rhesus macaques) have historically been the large animal model of choice for CWNA MCM efficacy studies. However, the swine model (esp. Gottingen minipig), has been explored recently as an alternate/supplemental model to the nonhuman primate for CWNA MCM efficacy studies (Langston and Myers, 2016; Murray et al., 2012; Saxena et al., 2011).

The expression and activity levels of AChE and BChE in the animal model are important factors to consider when extrapolating the results of CWNA MCM animal efficacy studies to humans. Therefore, it is not surprising that comparisons of the properties of AChE and BChE in common small animal models and humans are readily found in the literature (Worek and Thiermann, 2013). Although similar comparisons for large animal models have been reported (Luo et al., 2008), they are less common and generally more limited in scope, which confounds efforts to compare large animal species directly. To enable a direct comparison of the properties of AChE and BChE in relevant large animal models and humans, the authors set out to elucidate the basal activity of each enzyme in circulation as well as the properties of inhibition, reactivation and aging in humans, Yorkshire swine, Gottingen minipigs, African green monkeys, cynomolgus macaques, and both Indian and Chinese-origin rhesus macaques.

Materials and Methods

Chemicals. 5,5-dithio-bis-2-nitrobenzoic acid (DTNB), Acetylthiocholine iodide (ATC), S-butyrylthiocholineiodide (BTC), phosphate buffered saline (PBS), and pralidoxime chloride (2-PAM) were obtained from Sigma Aldrich (St. Louis, Mo.). All sarin was stored securely and in accordance with CWA storage policies until needed for testing. To preserve CWA purity, sarin was stored in individual vials until needed for testing.

Enzyme preparation. For the evaluation of basal AChE and BChE activity levels in the circulation, whole blood samples from 10 male and 10 female individuals from each species (i.e., human, Indian origin rhesus macaque, Chinese origin rhesus macaque, Yorkshire swine, Gottingen minipig, cynomolgus macaque, and African green monkey) were obtained from BioreclamationIVT (New York, N.Y.). Red blood cells (RBCs) and plasma were prepared from each sample using methods described previously (McGarry et al., 2013). For the evaluation of substrate hydrolysis, inhibition, reactivation, and aging, whole blood samples obtained from BioreclamationIVT were pooled from XX males from each species. RBCs and plasma were then prepared from each sample using methods optimized for the preparation of RBCs for kinetics experiments that were described previously (Andrews and Neises, 2012). All blood samples were collected using K3 EDTA as the anticoagulant.

Enzyme activity determination. AChE and BChE activity were determined using a spectrophotometric assay as described previously (Ellman et al., 1961; McGarry et al., 2013). Unless otherwise stated in the methods for each specific set of experiments, the final concentrations of the substrates and indicator were as follows: 1 mM ATC (for RBCs), 3 mM BTC (for plasma), and 0.5 mM DTNB (for both RBCs and plasma). All experiments were performed at 37° C. in PBS. absorbance readings were recorded every 20 seconds over a period of 10 minutes at 412 nm using a BioTek® Synergy™ HTX Multi-Mode Microplate Reader. Absorbance readings were initiated immediately after the addition of the substrate (ATC or BTC) for all experiments.

Substrate titrations. The substrate titration experiments were performed as described above except the final substrate concentrations ranged from 7.8 μM to 10 mM.

AChE and BChE inhibition assays. Concentration-response curves for each combination of matrix/species/CWNA were generated using CWNA concentrations ranging from 1.00E-04 M to 1.79E-12 M. Each reaction mixture was incubated for five minutes at 37° C. prior to the addition of substrate and DTNB. IC50 and IC90 values were calculated by nonlinear regression in GraphPad Prism® 7. For the determination of inhibition rate constants (ki), the IC90 concentration of each CWNA was incubated with each combination of matrix/species at 37° C. for various time intervals ranging from 0-15 min prior to the addition of substrate. The ki values were calculated as reported previously (Worek et al., 2004).

Oxime reactivation of AChE and BChE. RBC and plasma samples were incubated at 37° C. with the experimentally determined IC90 of VX or sarin for 15 minutes. Following incubation, unbound VX or sarin was removed by passing the reaction mixture through 7K MWCO Zeba spin desalting columns (Thermo Scientific; Waltham, Mass.). Following removal of unbound agent, agent-inhibited AChE and BChE were incubated at 37° C. with 500 μM DTNB for 5 min. Next, 2-PAM Cl or vehicle was added to the reaction mixture, immediately followed by addition of substrate. For all experiments that included 2-PAM, background resulting from oximolysis was accounted for during calculations.

Aging. RBC and plasma samples were incubated at 37° C. with the experimentally determined IC90 of VX or sarin for 15 minutes. Unbound VX or sarin was removed by passing the reaction mixture through 7K MWCO Zeba spin desalting columns (Thermo Scientific). Next, the inhibited enzyme was incubated at 37° C. for various time intervals (0-48 hr) before 2-PAM (50 μM) was added and enzyme activity was determined. The rate constants for aging (ka) and spontaneous reactivation (ks) were calculated using a nonlinear regression model in GraphPad Prism® 7.

Results

Cholinesterase Levels in Circulation

Compared to humans, AChE activity levels in circulation were similar in the nonhuman primates and significantly lower in swine (FIG. 1). For BChE, only Chinese origin rhesus macaque yielded results that were not significantly different from the human results (FIG. 1). Basal BChE activity in the plasma of Indian origin and cynomolgus macaques was significantly higher than that observed in humans, while African green monkeys and both swine displayed significantly lower values. The contribution of sex to AChE or BChE activity within each species was also evaluated, and significant differences were identified in Indian origin rhesus macaques for AChE and cynomolgus macaques for BChE (males displayed higher values in both cases; FIG. 1). When both enzymes are taken into account, the Chinese-origin rhesus macaque displayed circulating ChE levels most similar to those observed in humans.

Enzyme Inhibition

Results of the enzyme inhibition and inhibition kinetics experiments are shown in Table 1. In all species, AChE was more susceptible than BChE to inhibition by sarin and VX. Greater interspecies variability was observed for BChE (>10-fold variance between some ki values for the same nerve agent between species) than for AChE 5-fold variance between any two ki values for the same nerve agent between species). Of the species tested, human AChE and BChE were the most susceptible to inhibition by both nerve agents, although the ki and IC50 values were generally similar between humans and all of the nonhuman primates for AChE. In general, the enzyme inhibition results obtained for rhesus macaques were the most similar to human, and the results obtained for swine were the least similar.

TABLE 1
Nerve agent inhibition (IC50, M) and inhibition kinetics [ki (M−1min−1)] in multiple species (error represents ± the standard error of the mean)

| | Agent: VX | | | | Agent: Sarin | | | |
|---|---|---|---|---|---|---|---|---|
| | AChE | | BChE | | AChE | | BChE | |
| Species | IC₅₀ | kᵢ | IC₅₀ | kᵢ | IC₅₀ | kᵢ | IC₅₀ | kᵢ |
| Gottingen | 4.80 × 10⁻⁹ | 1.42 ± 0.11 × 10⁷ | 35.1 × 10⁻⁹ | 0.25 ± 0.06 × 10⁷ | 5.72 × 10⁻⁹ | 1.24 ± 0.08 × 10⁷ | 21.7 × 10⁻⁹ | 0.06 ± 0.01 × 10⁷ |
| Mini Pig | | | | | | | | |
| Yorkshire | 5.27 × 10⁻⁹ | 1.06 ± 0.10 × 10⁷ | 32.1 × 10⁻⁹ | 0.28 ± 0.04 × 10⁷ | 2.07 × 10⁻⁹ | 0.85 ± 0.05 × 10⁷ | 21.8 × 10⁻⁹ | 0.14 ± 0.02 × 10⁷ |
| Swine | | | | | | | | |
| (Weanling) | | | | | | | | |
| Indian | 1.82 × 10⁻⁹ | 2.56 ± 0.26 × 10⁷ | 32.4 × 10⁻⁹ | 1.36 ± 0.25 × 10⁷ | 0.80 × 10⁻⁹ | 1.81 ± 0.12 × 10⁷ | 8.61 × 10⁻⁹ | 0.50 ± 0.06 × 10⁷ |
| Origin | | | | | | | | |
| Rhesus | | | | | | | | |
| Macaque | | | | | | | | |
| Chinese | 1.89 × 10⁻⁹ | 2.97 ± 0.28 × 10⁷ | 40.0 × 10⁻⁹ | 1.44 ± 0.27 × 10⁷ | 0.59 × 10⁻⁹ | 1.52 ± 0.18 × 10⁷ | 6.74 × 10⁻⁹ | 0.57 ± 0.09 × 10⁷ |
| Origin | | | | | | | | |
| Rhesus | | | | | | | | |
| Macaque | | | | | | | | |
| Cynomolgus | 1.49 × 10⁻⁹ | 2.97 ± 0.21 × 10⁷ | 40.4 × 10⁻⁹ | 0.15 ± 0.02 × 10⁷ | 0.79 × 10⁻⁹ | 1.81 ± 0.15 × 10⁷ | 6.59 × 10⁻⁹ | 0.70 ± 0.06 × 10⁷ |
| Macaque | | | | | | | | |
| African | 1.95 × 10⁻⁹ | 2.65 ± 0.18 × 10⁷ | 40.6 × 10⁻⁹ | 0.13 ± 0.01 × 10⁷ | 1.68 × 10⁻⁹ | 1.70 ± 0.12 × 10⁷ | 19.1 × 10⁻⁹ | 0.34 ± 0.04 × 10⁷ |
| Green | | | | | | | | |
| Monkey | | | | | | | | |
| Human | 1.86 × 10⁻⁹ | 4.03 ± 0.31 × 10⁷ | 15.4 × 10⁻⁹ | 2.39 ± 0.43 × 10⁷ | 0.52 × 10⁻⁹ | 2.00 ± 0.16 × 10⁷ | 0.80 × 10⁻⁹ | 0.83 ± 0.14 × 10⁷ |

Aging

The inability to reactivate cholinesterases typically stems from a dealkylation reaction which occurs following the covalent bonding of an OP to the active site serine. This dealkylated, phosphylated serine adduct is no longer reactivatable by nucleophiles due to the resulting bond strength of the OP to the serine hydroxyl group and the local charges within the active site—specifically that the protonated histidine residue which constitutes of one-third of the catalytic triad, is no longer able to serve as a general base to assist in nucleophilic reactivation of the serine residue. The details of this reaction are well summarized by in both a more simplified cartoon format (Registry, 2007) and in great detail by Nachon and colleagues (Nachon et al., 2010). It should be noted, that the aging rate is a property specific to each individual OP and enzyme and not all OPs undergo an aging reaction.

As shown in Table 2 below, characteristic aging half times for sarin were observed with AChE, and, as expected, aging is not observed within 48 hours for any of the BChE activity nor VX results. There is one exception to these results, however, and that lies with the human BChE vs. sarin that displays an aging half-time of approximately 101/2 hours—approximately 2-fold slower than that of the AChE aging rate. While more work is needed to ascertain the basis for these observed results, it is possible that human BChE ages due to the presence of the rigid proline residue at position 285 within the acyl binding pocket of the enzyme. Humans are the only species evaluated that possess a proline at this position. This possibility and the differences of the human enzyme is discussed in greater detail in the corresponding manuscript.

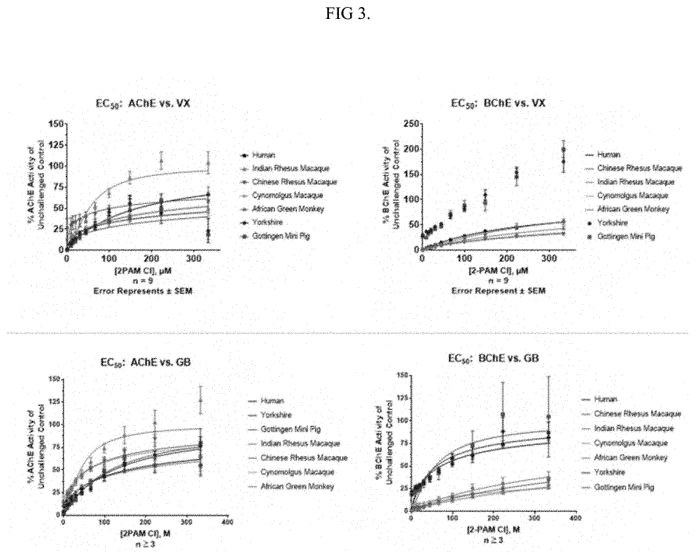

Most interestingly, however, was the observation of spontaneous reactivation of porcine BChE inhibited by sarin with a t1/2 of approximately 2 hours (FIG. 3). This is not observed with VX, however, and therefore this is a characteristic of this enzyme specific to inhibition by sarin (and/or other G-agents). To Applicant's knowledge, this has not been reported previously.

TABLE 2
Mean Aging Half-Times of Multiple Species

| | Mean Aging Half-Time (Hours) | |
|---|---|---|
| | Agent: Sarin | |
| Species | AChE | BChE |
| Gottingen Mini Pig | 9.2 | NA |
| Yorkshire Swine (Weanling) | 6.3 | NA |
| Indian Origin Rhesus Macaque | 7.4 | NA |
| Chinese Origin Rhesus Macaque | 5.3 | NA |
| Cynomolgus Macaque | 5.4 | NA |
| African Green Monkey | 8.2 | NA |
| Human | 5.0 | 10.6 |

Note: “NA” denotes no aging was observed within the 48-hour experimental timeframe.

Reactivation-EC50 Determination

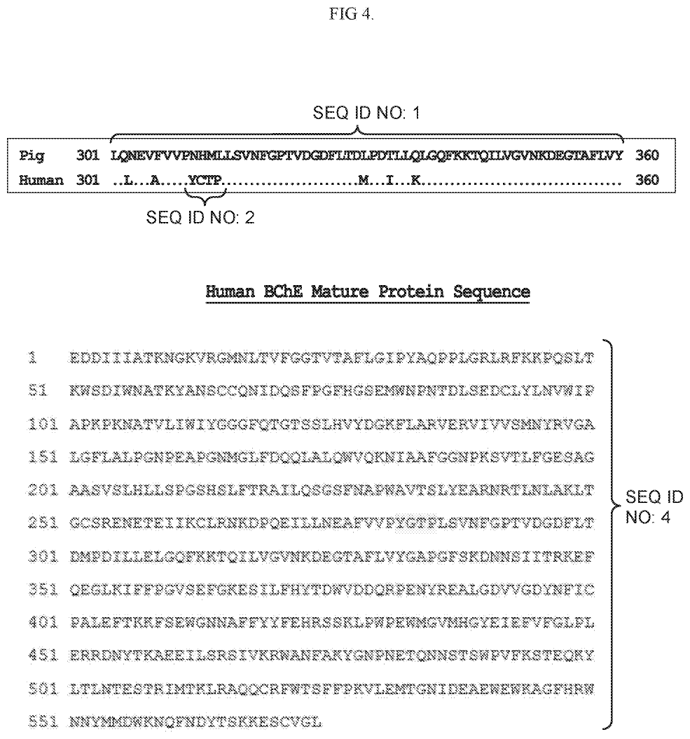

Calculated effective concentrations of 2-PAM Cl whereby inhibited enzyme is returned to 50% activity (EC50) were calculated. Results of the reactivation experiments for 2-PAM Cl vs. sarin- or VX-inhibited samples are shown in Table 3 and FIG. 4? for each species and enzyme. FIG. 4 is displayed as a representative figure for the various challenge/reactivation experiments. Additionally, FIG. 4 displays several interesting results: Firstly, the various primate species indicate that reactivation of VX-inhibited BChE by 2-PAM Cl is incomplete, resulting in an approximate 50% maximal activity following addition of the oxime. For AChE, with the exception of the Indian-origin rhesus macaque, all other species display incomplete reactivation. Secondly, an apparent biphasic and synergistic effect of 2-PAM Cl with BChE vs. VX is observed as activity returns to >100% of the unchallenged control enzyme. Interestingly, this is not observed following a sarin challenge—although activity is returned to ˜100% in the two pig species and human BChE. It is unclear at this moment why the pig BChE behaves in this manner. Without intending to be limited by theory, one may speculate that this result might stem from a substrate activation-like event which has been reported previously for human BChE, with the butyrylthiocholine substrate (Lockridge et al., 1997).

TABLE 3
EC50 (μM) Determination of 2-PAM Cl for Multiple Species versus VX or Sarin

| | Agent: VX | | Agent: Sarin | |
|---|---|---|---|---|
| Species | AChE | BChE | AChE | BChE |
| Yorkshire Swine | 177.8 ± 31.4 | 25.3 ± 4.4 | 126.9 ± 9.2 | 60.5 ± 7.7 |
| Gottingen Mini Pig | 236.6 ± 28.6 | 30.6 ± 5.5 | 105.9 ± 11.4 | 57.2 ± 13.5 |
| Indian Rhesus Macaque | 52.3 ± 3.6 | 458.5 ± 27.8 | 40.4 ± 4.2 | 1142 ± 92.2 |
| Chinese Rhesus Macaque | 95.2 ± 30.9 | 825.4 ± 62.1 | 65.9 ± 8.3 | 775.7 ± 99 |
| Cynomolgus Macaque | 204.8 ± 30.1 | 735.2 ± 41.9 | 69.1 ± 7 | 3507 ± 12091 |
| African Green Monkey | 221.6 ± 22.1 | 273.8 ± 19.4 | 208.2 ± 13.9 | 582 ± 107.1 |
| Human | 167.1 ± 17.4 | 263.2 ± 6.2 | 142.7 ± 22.8 | 72.2 ± 5.7 |

Development of animal models for conditions that affect humans has been at the forefront of therapeutic development for decades. Both small and large animal models for human therapeutic development have their place in the field and understanding the limitations of a given model is imperative for the interpretation of the results generated using the model. For MCMs seeking approval under the FDA's Animal Rule, it is generally recommended that MCM efficacy be demonstrated in more than one species that is expected to react with a response predictive of the human response (2015). NHPs are an accepted large animal model for biomedical research due to their similarities to humans, and rhesus macaques have been the most frequently used large animal model for advanced development of CWNA MCMs. However, there are a number of important factors that are unrelated to biology that do not favor the continued use of NHPs (particularly Indian origin rhesus macaques) for CWNA MCM research. These factors include availability, public perception, political pressure, and the high cost associated with working with NHPs due to procurement and specialized housing, animal husbandry, and training requirements. In light of these factors, swine models (esp. Göttingen minipig) have been explored as an alternative to rhesus macaque as a large animal model for CWNA MCM efficacy evaluation.

Pigs have been used in biomedical research for many years and many of their organ systems (esp. skin) share similarities with humans. Porcine models have been shown to be well suited as human surrogates for skin permeability (Bartek et al., 1972, Simon, G. A. and H. I. Maibach, The pig as an experimental animal model of percutaneous permeation in man: qualitative and quantitative observations—an overview. Skin Pharmacol Appl Skin Physiol, 2000. 13(5): p. 229-34.) and decontamination studies (Simon and Maibach, 2000; Bartek et al., 1972; Taysse, L., et al., Skin decontamination of mustards and organophosphates: comparative efficiency of RSDL and Fuller's earth in domestic swine. Hum Exp Toxicol, 2007. 26(2): p. 135-41). In studies that are relevant to CWNA research, pigs were utilized for the evaluation of the dermal penetration and pharmacokinetics of cholinesterase-inhibiting pesticides. (Qiao, G. L. and J. E. Riviere, Significant effects of application site and occlusion on the pharmacokinetics of cutaneous penetration and biotransformation of parathion in vivo in swine. J Pharm Sci, 1995. 84(4): p. 425-32, Baynes, R. E., K. B. Halling, and J. E. Riviere, The influence of diethyl-m-toluamide (DEET) on the percutaneous absorption of permethrin and carbaryl. Toxicol Appl Pharmacol, 1997. 144(2): p. 332-9.) Gottingen minipigs Unfortunately, swine are generally considered challenging animals to work with due to their large size and temperament. As such, a large amount of MCM research has had to adopt the use of anesthesia (O'Koren et al., 2013) to control swine and decrease the amount of physical stress placed on the animal. This poses problems for studies involving CWNAs, as anesthetic compounds inhibit cholinesterase activity (Honavar et al., 2011) which can potentially further confound the translatable results of these studies to humans. In addition to issues with anesthesia, notable differences have been observed in AChE-oxime interactions between swine and humans. Compared to humans, swine AChE shows a lower sensitivity towards select oximes, as well as slower reactivation and aging (Honavar et al., 2014). In addition to kinetic differences, as observed by Phillips and colleagues (and as confirmed within the data presented herein) swine also have lower constitutive levels of BChE which limits their effectiveness as models used in BChE bioscavenger studies (Phillips et al., 2004). Lastly, it has been shown that certain swine models have greatly increased paraoxonase 1 (PON1) levels compared to humans (D'Agostino et al., 1999). PON1 is a promiscuous enzyme that possesses the capability of hydrolyzing various CWNAs. All of these factors suggest swine as being a less-than-ideal large animal model for MCM research for CWNAs.

As such, the objective of this study was to perform a comprehensive comparison of human AChE and BChE to the cholinesterases derived from six large animal research models. The purpose of this project was to assess the cholinesterase profiles in an effort to inform species selection of large animal models for medical countermeasure development against CWNAs.

To evaluate the question of which species is best for MCM development and ultimately, translation of the data obtained to expected human results, levels of circulating AChE and BChE, as well as enzyme characteristics following inhibition by the CWNAs VX and sarin were evaluated in isolated RBC membranes and plasma from multiple species and compared to human data. Of the enzyme characteristics evaluated, many commonalities between the species were observed. There were, however, several notable variations between the species. While the Indian-origin rhesus macaque is commonly used for nerve agent research, data described above indicates that that Chinese-origin rhesus macaque may actually be a more suitable model based upon circulating cholinesterase levels and minimal differences observed between males and females. Additionally, differences in the inhibition rates of each of the enzymes can be observed between species. However, the range and fold-differences between them are not likely biologically relevant. Therefore, it can be assumed that the ChEs from each of the species bind the agents and the enzymes are rapidly inhibited through a covalent interaction at the active site serine as has been shown numerous times before. Additionally, aging of BChE was only observed with the human enzyme. Without intending to be limited by theory, it is possible that human BChE ages due to the presence of the rigid proline residue at position 285 within the acyl binding pocket of the enzyme. Humans are the only species evaluated that possess a proline at this position.

The most striking results, however, were the results from the swine experiments. Swine displayed significantly lower levels of both AChE and BChE as compared to humans. The lower BChE levels severely limit the animal's bioscavenging capability allowing them to succumb to nerve agent toxicity more rapidly. Additionally, however, a relatively rapid spontaneous reactivation of BChE following inhibition by sarin observed in the swine models. The initial explanation for this phenomenon related to the elevated levels of the promiscuous enzyme, paroxonase 1 (PON1) in the plasma of swine (Ceron et al., 2014). The spontaneous reactivation of plasma BChE in the swine, however, is not due to elevated PON1 activity, but rather a variation of the residues within and adjacent to the acyl binding pocket of the enzyme. To compound this observation, a seemingly synergistic effect of BChE with 2 PAM Cl following VX inhibition is observed as BChE activity exceeds 100% of basal, unchallenged activity. It may be that, without intending to be limited by theory, this result might stem from a substrate activation-like event which has been reported previously for human BChE, with the butyrylthiocholine substrate (Lockridge et al., 1997).

### EXAMPLE 1 REFERENCES

Andrews, J. A., Neises, K. D., 2012. Cells, biomarkers, and post-traumatic stress disorder: evidence for peripheral involvement in a central disease. J Neurochem 120, 26-36.
Aygun, D., Erenler, A. K., Karatas, A. D., Baydin, A., 2007. Intermediate syndrome following acute organophosphate poisoning: correlation with initial serum levels of muscle enzymes. Basic Clin Pharmacol Toxicol 100, 201-204.
Cannard, K., 2006. The acute treatment of nerve agent exposure. J Neurol Sci 249, 86-94.
Ceron, J. J., Tecles, F., Tvarijonaviciute, A., 2014. Serum paraoxonase 1 (PON1) measurement: an update. BMC Vet Res 10, 74.
D'Agostino, P. A., Hancock, J. R., Provost, L. R., 1999. Packed capillary liquid chromatography-electrospray mass spectrometry analysis of organophosphorus chemical warfare agents. J Chromatogr A 840, 289-294.
Eddleston, M., Buckley, N. A., Eyer, P., Dawson, A. H., 2008. Management of acute organophosphorus pesticide poisoning. Lancet 371, 597-607.
Ellman, G. L., Courtney, K. D., Andres, V., Jr., Feather-Stone, R. M., 1961. A new and rapid colorimetric determination of acetylcholinesterase activity. Biochem Pharmacol 7, 88-95.
Eyer, P., 2003. The role of oximes in the management of organophosphorus pesticide poisoning. Toxicol Rev 22, 165-190.
Honavar, J., Bradley, E., Bradley, K., Oh, J. Y., Vallejo, M. O., Kelley, E. E., Cantu-Medellin, N., Doran, S., Dell'italia, L. J., Matalon, S., Patel, R. P., 2014. Chlorine gas exposure disrupts nitric oxide homeostasis in the pulmonary vasculature. Toxicology 321, 96-102.
Honavar, J., Samal, A. A., Bradley, K. M., Brandon, A., Balanay, J., Squadrito, G. L., MohanKumar, K., Maheshwari, A., Postlethwait, E. M., Matalon, S., Patel, R. P., 2011. Chlorine gas exposure causes systemic endothelial dysfunction by inhibiting endothelial nitric oxide synthase-dependent signaling. Am J Respir Cell Mol Biol 45, 419-425.
Jones, M. E., Lebonville, C. L., Paniccia, J. E., Balentine, M. E., Reissner, K. J., Lysle, D. T., 2018. Hippocampal interleukin-1 mediates stress-enhanced fear learning: A potential role for astrocyte-derived interleukin-1beta. Brain Behav Immun 67, 355-363.
Li, B., Duysen, E. G., Carlson, M., Lockridge, O., 2008. The butyrylcholinesterase knockout mouse as a model for human butyrylcholinesterase deficiency. J Pharmacol Exp Ther 324, 1146-1154.
Lockridge, O., Blong, R. M., Masson, P., Froment, M. T., Millard, C. B., Broomfield, C. A., 1997. A single amino acid substitution, Gly117His, confers phosphotriesterase (organophosphorus acid anhydride hydrolase) activity on human butyrylcholinesterase. Biochemistry 36, 786-795.
Lorke, D. E., Kalasz, H., Petroianu, G. A., Tekes, K., 2008. Entry of oximes into the brain: a review. Curr Med Chem 15, 743-753.
Luo, C., Tong, M., Maxwell, D. M., Saxena, A., 2008. Comparison of oxime reactivation and aging of nerve agent-inhibited monkey and human acetylcholinesterases. Chem Biol Interact 175, 261-266.
Manoharan, I., Boopathy, R., Darvesh, S., Lockridge, O., 2007. A medical health report on individuals with silent butyrylcholinesterase in the Vysya community of India. Clin Chim Acta 378, 128-135.
McGarry, K. G., Bartlett, R. A., Machesky, N. J., Snider, T. H., Moyer, R. A., Yeung, D. T., Brittain, M. K., 2013. Evaluation of HemogloBind treatment for preparation of samples for cholinesterase analysis. Adv Biosci Biotechnol 4, 1020-1023.
Mo, Y., Chen, J., Schlueter, C. F., Hoyle, G. W., 2013. Differential susceptibility of inbred mouse strains to chlorine-induced airway fibrosis. Am J Physiol Lung Cell Mol Physiol 304, L92-102.
Mosley, M., Shah, C., Morse, K. A., Miloro, S. A., Holmes, M. M., Ahern, T. H., Forger, N. G., 2017. Patterns of cell death in the perinatal mouse forebrain. J Comp Neurol 525, 47-64.
Nachon, F., Carletti, E., Worek, F., Masson, P., 2010. Aging mechanism of butyrylcholinesterase inhibited by an N-methyl analogue of tabun: implications of the trigonal-bipyramidal transition state rearrangement for the phosphylation or reactivation of cholinesterases. Chem Biol Interact 187, 44-48.
O'Koren, E. G., Hogan, B. L., Gunn, M. D., 2013. Loss of basal cells precedes bronchiolitis obliterans-like pathological changes in a murine model of chlorine gas inhalation. Am J Respir Cell Mol Biol 49, 788-797.
Phillips, R. J., Burdick, M. D., Hong, K., Lutz, M. A., Murray, L. A., Xue, Y. Y., Belperio, J. A., Keane, M. P., Strieter, R. M., 2004. Circulating fibrocytes traffic to the lungs in response to CXCL12 and mediate fibrosis. J Clin Invest 114, 438-446.
Radic, Z., Pickering, N. A., Vellom, D. C., Camp, S., Taylor, P., 1993. Three distinct domains in the cholinesterase molecule confer selectivity for acetyl- and butyrylcholinesterase inhibitors. Biochemistry 32, 12074-12084.
Reed, B. A., Sabourin, C. L., Lenz, D. E., 2017. Human butyrylcholinesterase efficacy against nerve agent exposure. J Biochem Mol Toxicol 31.
Registry, A.f.T.S.a.D., 2007. Cholinesterase Inhibitors: Including Insecticides and Chemical Warfare Nerve Agents.
Snoy, P. J., 2010. Establishing efficacy of human products using animals: the US food and drug administration's “animal rule”. Vet Pathol 47, 774-778.
Thiermann, H., Worek, F., Kehe, K., 2013. Limitations and challenges in treatment of acute chemical warfare agent poisoning. Chem Biol Interact 206, 435-443.
Vellom, D. C., Radic, Z., Li, Y., Pickering, N. A., Camp, S., Taylor, P., 1993. Amino acid residues controlling acetylcholinesterase and butyrylcholinesterase specificity. Biochemistry 32, 12-17.
Worek, F., Aurbek, N., Wetherell, J., Pearce, P., Mann, T., Thiermann, H., 2008. Inhibition, reactivation and aging kinetics of highly toxic organophosphorus compounds: pig versus minipig acetylcholinesterase. Toxicology 244, 35-41.
Worek, F., Thiermann, H., Szinicz, L., Eyer, P., 2004. Kinetic analysis of interactions between human acetylcholinesterase, structurally different organophosphorus compounds and oximes. Biochem Pharmacol 68, 2237-2248.
Worek, F., Thiermann, H., Wille, T., 2016. Oximes in organophosphate poisoning: 60 years of hope and despair. Chem Biol Interact 259, 93-98.
Zhang, J., Dai, H., Deng, Y., Tian, J., Zhang, C., Hu, Z., Bing, G., Zhao, L., 2015. Neonatal chlorpyrifos exposure induces loss of dopaminergic neurons in young adult rats. Toxicology 336, 17-25.

### Example 2. A Novel, Human-Porcine Hybrid Butyrylcholinesterase Catalytically Degrades the Chemical Warfare Nerve Agent, Sarin

Chemical warfare nerve agents (CWNAs) present a global threat to both military and civilian populations. The acute toxicity of CWNAs stems from their ability to strongly inhibit acetylcholinesterase (AChE). This inhibition can lead to uncontrolled cholinergic cellular signaling, resulting in cholinergic crisis and, ultimately, death. While the current FDA-approved standard of care is moderately effective when administered early, development of novel treatment strategies is necessary. Butyrylcholinesterase (BChE) is an enzyme which displays a high degree of structural homology to AChE. Unlike AChE, BChE appears to be a non-essential enzyme. BChE, however, appears to primarily serve as a bioscavenger of toxic esters due to its ability to accommodate a wide variety of substrates within its active site. Like AChE, BChE is also readily inhibited by CWNAs. Due to its high affinity for binding CWNAs, and that null-BChE yields no apparent health effects, exogenous BChE has been explored as a candidate therapeutic for CWNA intoxication. Despite years of research, minimal strides have been made to develop a catalytic bioscavenger. Further, BChE is only in early clinical trials as a stoichiometric bioscavenger of CWNAs, and large quantities must be administered to treat CWNA toxicity. Here Applicant describes previously unidentified mutations to residues within and adjacent to the acyl binding pocket of BChE that confer catalytic degradation of the CWNA, sarin. These mutations may provide a novel therapeutic to combat CWNA intoxication.

Organophosphorus (OP) chemical warfare nerve agents (CWNAs), such as sarin and VX, are some of the most potent neurotoxicants known. Many pesticides also belong to this class of chemicals but are generally much less toxic. The primary mechanism of OP toxicity arises from inhibition of acetylcholinesterase (AChE) via phosphylation of the active serine site¹⁻⁴. This phosphylation event can have devastating consequences to a poisoned individual.

In cellular signaling involving cholinergic neurons, the neurotransmitter acetylcholine (ACh) is packaged and released into the synaptic cleft where it binds to and activates ACh-receptors, continuing a downstream signaling event. To attenuate the signaling event, AChE hydrolyzes excess ACh, thereby terminating neuronal signaling. Thus, AChE is an essential regulator of neuronal cellular signaling. Inhibition of AChE results in the accumulation of ACh, leading to over-stimulation of ACh receptors and may ultimately lead to cholinergic crisis. The continuous stimulation of cholinergic neurons and/or skeletal muscles may result in seizures, paralysis, and ultimately respiratory failure and death⁵. The current FDA-approved/fielded standard of care for OP poisoning includes the use of three drugs: 1) a muscarinic antagonist (e.g., atropine); 2) an AChE reactivator (e.g., pralidoxome); and 3) a benzodiazepine (e.g. diazepam), to control seizures.

Butyrylcholinesterase (BChE) exhibits a high degree of structural similarity and sequence homology to AChE^{6; 7}, and, like AChE, can hydrolyze the neurotransmitter acetylcholine. Notably, BChE is also inhibited by CWNAs. BChE is believed to have arisen early in the evolution of vertebrates due to a gene duplication event, and the structural similarities of the two enzymes are striking⁸. Most notably, the catalytic triad of BChE, like AChE, resides at the bottom of a 20 Å deep gorge that is lined with aromatic amino acid residues. However, while AChE has fourteen aromatic residues lining its gorge which facilitates pi-pi stacking interactions, BChE retains only eight⁹. Interestingly, the endogenous function of BChE remains largely unknown as individuals deficient in active BChE appear to lead normal, healthy lives¹⁰. BChE, however, is thought to serve in a number of biological processes, including neuronal cellular development and neuronal signaling and also appears to have a role in bronchial airway smooth muscle function^{11; 12}.

One significant function of BChE is the ability to serve as a bioscavenger of toxic esters. The difference in the volume of the active sites between the two enzymes allows BChE to hydrolyze a wider variety of substrates and thus serve in this capacity¹³.

The use of exogenous human BChE as a prophylactic bioscavenger for CWNA poisoning has been evaluated in animal studies¹¹. Indeed, exogenous BChE is protective against OP poisoning, although large amounts of BChE are required—for example, more than 20 mg/kg of BChE was required to protect guinea pigs against a 2.5× LD50 percutaneous challenge of VX^{14; 15}. With these promising results, BChE is being evaluated in early clinical trials. However, the binding of CWNAs to BChE is stoichiometric and irreversible (i.e., one molecule of BChE is required to remove one molecule of CWNA). Thus, the required dose of BChE imposes a challenge for OP protection due to limitations on how much exogenous protein may be administered safely. The volume of human serum required for purification and the cost associated with the purification pose other challenges for BChE as a medical countermeasure^{11; 16-19}.

Mutagenesis of BChE to create a catalytic or self-reactivating bioscavenger has been extensively explored¹⁶. Lockridge and colleagues discovered that a point mutation (G117H) within the oxyanion hole confers organophosphate hydrolase characteristics to BChE²⁰. This discovery led to two decades of research regarding the creation and optimization of catalytic and pseudocatalytic (oxime-assisted) bioscavengers based on wild-type human BChE. Relatively recent developments involving several species and various mutations indicates that increased flexibility within the acyl loop of both pig BChE and bovinated-human BChE appears to promote auto-reactivation following inhibition by VX or chloropyrifos oxon²¹⁻²⁴. To date, however, neither the G117H mutation alone, nor in combination with other mutations, has yielded an enzyme that displays sufficient activity required for a catalytic bioscavenger (i.e. k_{cat}/K_{M}>10⁵ M⁻¹s⁻¹;¹⁷) while retaining sufficient binding affinity to surpass the effectiveness of wildtype BChE as a medical countermeasure.

Herein, motivated by the catalytic activity of porcine BChE, Applicant implemented some specific modifications to the human isoform, and then expressed this novel human-porcine hybrid BChE. The resulting enzyme appears to have the remarkable ability to catalytically degrade the CWNA sarin, while retaining near wild-type binding affinity.

Materials and Methods

Chemicals. 5,5-dithio-bis-2-nitrobenzoic acid (DTNB), acetylthiocholine iodide (ATC), S-butyrylthiocholine iodide (BTC), phosphate buffered saline (PBS), 3-(N-morpholino) propanesulfonic acid, 4-morpholinepropanesulfonic acid (MOPS), and pralidoxime chloride (2-PAM Cl) were obtained from Sigma Aldrich (St. Louis, Mo.). All tests involving authentic chemical agents were performed at the Battelle Biomedical Research Center (BBRC) located in West Jefferson, Ohio. The BBRC is certified to work with chemical surety material under a Provisioning Agreement with oversight by the U.S. Army Materiel Command (AMC; Provisioning Agreement Battelle-1). Wherever applicable and required, the reporting requirements for this agreement were followed. All quantities of sarin used during this testing were synthesized at Battelle's Hazardous Materials Research Center (HMRC) under Chemical Weapons Convention program guidelines, with accountability through the U.S. AMC. The sarin originated from the same synthesis lot. All sarin was stored in accordance with BBRC security and CWA storage policies until needed for testing. To preserve CWA purity, sarin was stored in individual vials until needed for testing.

BLAST® Sequence Alignments. Using the National Center for Biotechnology Information (NCBI) website, a BLAST® sequence alignment was performed to compare the amino acid sequences of BChE from five species. The species that were compared and their corresponding accession numbers were as follows: Human (Homo sapiens; P06276.1), Pig (Sus scrota; NP_001344438.1), African Green Monkey (Chlorocebus sabaeus; XP_007970394.1), Rhesus Macaque (Macaca mulatta; XP_002808379.2), and the Crab-eating/Cynomolgus macaque (Macaca fascicularis; NP_001306299.1).

Plasmid Construction. The gene encoding human BChE was purchased from MyBiosource and cloned into the pTT expression vector²⁵ using the HindIII and AfeI restriction sites. This vector was selected for its high level of gene transfer and protein expression in transiently transfected human embryonic kidney, HEK 293 EBNA1 cells (HEK293E; ATCC: CRL-10852)²⁶. As required, site directed mutagenesis was performed using a modified QuikChange® reaction whereby Q5® High-Fidelity DNA Polymerase, 5× Q5 Reaction Buffer, and Q5 High GC Enhancer supplement (New England BioLab Cat. No. M0491S) were utilized. The QuikChange® primer design tool (available at Agilent's website; https://www.chem.agilent.com/store/primerDesignProgram.jsp) and cycling conditions described within the QuikChange® protocol were used as described.

Enzyme production and purification. Multiple recombinant BChEs were transiently expressed and purified in HEK293E cells. Briefly, HEK293E cells were grown in 75 cm² tissue culture flasks with vent caps containing 20 mL of Dulbecco's Modified Eagle's medium supplemented with 10% fetal bovine serum and 8 mM L-glutamine to ˜70% confluence. Separate flasks (two per construct, 40 mL total) were transiently transfected using a Lipofectamine® 3000 Reagent kit (Invitrogen) with 20 μg DNA. Expression media was collected 7 days post-transfection and centrifuged at 100×g for 5 minutes to remove cellular debris. The resulting supernatant was diluted ≥2-fold in 1× Phosphate Buffered Saline, pH 7.4+500 mM NaCl+10 mM imidazole and subsequently purified. Briefly, using a gravity column, the diluted supernatant was passed over an 800 bed volume of Ni-NTA resin (Thermo Fisher Scientific) equilibrated with 1× Phosphate Buffered Saline, pH 7.4+500 mM NaCl+10 mM imidazole. Columns were washed once with ten column volumes (CVs) 1× Phosphate Buffered Saline, pH 7.4+500 mM NaCl+10 mM imidazole and once with 1× Phosphate Buffered Saline, pH 7.4+500 mM NaCl+25 mM imidazole. The protein was eluted with 1× Phosphate Buffered Saline, pH 7.4+500 mM NaCl+100 mM imidazole and 1 column volume (CV) fractions were collected. Fraction #2 of each purification was confirmed via activity assays and protein gel analysis to contain the highest amount of the desired product and therefore was used for each construct in the subsequent experiments.

Protein Quantification. To determine the amount of purified protein produced from the transient transfection of HEK293E, a bicinchoninic acid assay (BCA) was used. Briefly, using a Micro BCA™ Protein Assay Kit (Thermo Fisher Scientific), working reagent and BCA standards were prepared per manufacturer instructions. Experimental protein samples were diluted 1:10 in 1×PBS, pH 7.4. Each test plate was prepared by adding 150 μL of standard or protein samples to each appropriate well, followed by the addition of 150 μL of working reagent. The test plate was then incubated at 37° C. for 120±5 minutes with gentle shaking. At the end of the incubation period, the plate was cooled for a minimum of 5 minutes at room temperature. The plate was analyzed by measuring the absorbance at 562 nm within 15 minutes of the incubation stop time using a BioTek® Synergy™ HTX Multi-Mode Microplate Reader. Unknown protein concentrations were calculated by interpolating the values from the standard curve.

Western blotting. To confirm the presence of the various, purified BChEs, anti-histidine Western blotting was performed. Experiments were performed by first denaturing and separating the second elution from the purification aliquots under denaturing conditions using a 4-12% gradient polyacrylamide gel (Thermo Fisher Scientific). Transfer to the polyvinylidene difluoride (PVDF) membrane was performed using an iBlot™ 2 (Life Technologies) per manufacturer's instructions. Immunoblotting was performed as follows: Following transfer, the membrane was rinsed in PBS, pH 7.4+0.1% Tween-20 (PBST) and blocked overnight at 2-8° C. with 5% non-fat milk (prepared in PBST). The membrane was rinsed three times with PBST and the primary antibody (mouse anti-histidine IgG; Invitrogen Cat. No. MA1-21315) was prepared in 1% milk in PBST at a 1:10,000 dilution and added to the membrane. The membrane was incubated in the presence of the primary antibody for >3 hours at 2-8° C. The membrane was rinsed three times with PBST and the secondary antibody (Horseradish peroxidase conjugated Goat anti-mouse IgG; Invitrogen Cat. No. 62-6520) was prepared in 1% milk in PBST at a 1:20,000 dilution and added to the membrane. The membrane was incubated in the presence of the secondary antibody for 1 hour at room temperature. The membrane was rinsed three times with PB ST. Finally enhanced chemiluminescent (ECL) substrate (Thermo Fisher Scientific) was added to the membrane and incubated at room temperature for 5 minutes prior to imaging.

Silver Stain. To observe the purity at each step of the immobilized metal affinity chromatography purification scheme, each aliquot from the various purification steps was denatured and then separated under denaturing conditions using a 6% polyacrylamide gel. To visualize the protein bands, a silver stain was performed. To perform the silver stain, each gel was rinsed in deionized water following completion of the PAGE. Fixation solution (30% ethanol, 10% acetic acid) was then added to each of the gels and incubated at room temperature for >18 hours with mild shaking. Gels were then rinsed four times (twice in 20% ethanol and twice in deionized water) for 10 minutes each rinse. Sensitizing solution (0.8 mM Sodium thiosulfate) was added to each gel and incubated with mild shaking for 1 minute at room temperature. Gels were rinsed twice in deionized water for 1 minute to remove excess sensitizing solution. The gels were then stained with 12 mM (2.04 mg/mL) AgNO₃ for 20-120 minutes. The gels were again briefly rinsed with deionized water (10-30 seconds). Developer solution (3% w/v potassium carbonate, 10% w/v sodium thiosulfate, and 0.025% formalin) was added and the gels were allowed to react until bands became clearly visible (˜5-10 minutes). The reactions were stopped using stop solution (4% w/v Tris Base, 2% v/v acetic acid) prior to imaging.

Enzyme activity assays. The rate of hydrolysis of varying concentrations of the chromogenic substrate, butyrylthiocholine iodide (BTC), was evaluated for the recombinant BChE enzymes in 10 mM MOPS buffer, pH 7.4 (unless otherwise indicated). The final BTC concentration was 3 mM, except for the substrate titration experiments, in which case the final BTC concentrations ranged from 7.8 μM to 10 mM. A concentration of 0.50 mM DTNB was used for all enzyme activity assays. BChE activity was determined spectrophotometrically using a method similar to that described previously²⁷. Absorbance readings were captured at 412 nm using a BioTek® Synergy™ HTX Multi-Mode Microplate Reader and enzyme activity was calculated using Beer's Law, where A=εlc. All experiments were performed at 37° C.

Enzyme inhibition. Enzyme inhibition was studied using the potent chemical warfare nerve agent, sarin. Recombinant BChEs were inhibited with varying concentrations of sarin ranging from 1.00×10⁻⁴ M to 1.79×10⁻¹² M in PBS (pH 7.4) or in 10 mM MOPS buffer (pH 7.4) to produce an inhibition curve. After the addition of agent, samples were incubated at 37° C. for >5 minutes. Enzymatic activity was monitored as described above. The concentrations which inhibited 50% and 90% of enzymatic activity (IC₅₀ and IC₉₀ respectively) were calculated using GraphPad Prism® 7.

Inhibition rate constant determination. Recombinant BChE samples were incubated with the experimentally determined IC₉₀ sarin concentration at pH 7.4 and 37° C. for various time intervals (0-15 minutes) prior to assessing enzymatic activity as described above. The inhibition rate constant, kᵢ, was calculated as described previously²⁸.

Aging. Recombinant BChE was incubated at 37° C. with the experimentally determined IC₉₀ of sarin for 15 minutes at pH 7.4. Unbound nerve agent was removed by passing the reaction mixture through 7K MWCO Zeba™ spin desalting columns (Thermo Fisher Scientific Cat. No. 89894). Following removal of excess, unbound nerve agent, the inhibited enzymes were incubated at 37° C. with an aliquot being removed at various time intervals (0-48 hr). 2-PAM Cl (50 μM) was added and enzymatic activity was determined using the Ellman's method described above.

GC-HRMS. To generate samples for GC-HRMS analysis, porcinated human BChE was challenged with 10, 167, or 500-fold molar excess sarin at 37° C. and aliquots were removed at T=0, 2, and 5 hours post-challenge and precipitated with ethyl acetate. Samples were stored at −70° C. prior to analysis. The sarin analysis was performed using a gas chromatographic (GC) system coupled with a Thermo DFS™ magnetic sector high resolution mass spectrometer (FIRMS). Ionization was accomplished in electron impact (EI) ionization mode. The ion calibration gas was PFTBA, and the lock mass used was 99.99306 Da. The outer source of the MS was maintained at 250° C. and the injection volume was 2 μL. The GC column was a CP Sil-5 column with a 0.25-mm internal diameter and 1.0 μm film thickness (Agilent, Santa Clara, Calif., USA). Helium carrier gas was maintained at a constant flow of 1.0 mL/min, and the transfer line was set at 280° C. The GC program began at 50° C. for 2 min, ramped to 70° C. at 20° C./min and was held for 4.5 min, ramped to 80° C. at 4° C./min, then ramped to 280° C. at 30° C./min, where it was held for 5 min. The total GC run time was 21.1 min. Supplemental Table 51 contains a summary of the instrumental parameters and a list of the masses that were monitored during the analysis.

Supplemental TABLE S1
GC-HRMS Parameters

| Mass Spectrometer | Thermo DFS | | |
|---|---|---|---|
| GC | Thermo Trace Ultra w/TriPlus Autosampler | | |
| Data Acquisition Software | Xcalibur 2.0 | | |
| Data Reduction Software | Waters MassLynx 4.0 | | |
| Column | Agilent CP Sil-5 - 30 m × 0.25 mm I.D. × 1.0 μm film | | |
| Temperature Program | Rate (° C./min) | Temperature (° C.) | Hold (min) |
| | 0 | 50 | 2.0 |
| | 20 | 70 | 4.5 |
| | 4 | 80 | 0 |
| | 30 | 280 | 5 |
| Total Run Time | 21.1 min | | |
| Carrier Gas/flow | Helium/1.0 mL/min constant flow | | |
| Injection Mode | Splitless | | |
| Injector Temperature | 250° C. | | |
| Split Flow/Splitless Time | 30 mL/min/1.0 min | | |
| Injection Volume | 2 μL | | |
| Autosampler Temperature | Ambient | | |
| Transfer Line Temperature | 280° C. | | |
| Ion Source | Electron Ionization (40 eV) | | |
| Polarity | Positive | | |
| Source Temperature | 250° C. | | |
| Scan Type | MID - Lock Mass | | |
| Acquisition Time | 6.5-10.0 min | | |
| | | | Approximate Retention |
| Monitored Ions | Compound | Mass (Da) | Time (minutes) |
| | Sarin Primary | 99.00057 | 7.88 |
| | Sarin Secondary | 125.01622 | 7.88 |
| | PFTBA Lock Mass | 99.99306 | NA |
| | PFTBA Calibration | 130.99147 | NA |

Working stock solutions of sarin were prepared from the sarin stock solution in ethyl acetate for use in preparation of calibration standards and quality control (QC) samples. A calibration (standard) curve was analyzed at concentrations of 0.2, 0.4, 1.0, 2.0, 5.0 and 20.0 ng/mL. A weighted linear regression curve using 1/x as the weighting factor, with x being the concentration of sarin spiked in ng/mL, and y being the sarin peak area, was used to calculate the correlation coefficient (r=0.971). The formula for linear regression was used as follows: y=mx+b.

LC-MS/MS. The analysis of sarin and its hydrolysis product, isopropyl methylphosphonic acid (IMPA), was performed using an ultra-pressure liquid chromatographic (UPLC) system coupled with a Waters TQ-XS quadrupole mass spectrometer (MS/MS). Data was collected with MassLynx 4.2 (Waters, Milford, Mass., USA) using multiple reaction monitoring (MRM) for each of the ion transitions summarized in Supplemental Table S2. The electrospray ionization source was operated in positive ion mode and under the following parameters: curtain gas=nitrogen at 1,000 L/hr; collision gas=argon at 0.15 mL/min; ion spray voltage=1 kV; temperature=110° C. The collision energy and cone voltage were optimized for each transition and are reported in Supplemental Table S2, along with the rest of the instrumental parameters. The timepoint sample aliquots (15 μL) were separated by reversed-phase chromatography using a Phenomenex Prodigy ODS HPLC column. Mobile phases were 0.1% formic acid in HPLC grade water (mobile phase A) and 0.1% formic acid in acetonitrile (mobile phase B). The gradient profile used is summarized in Supplemental Table S2 at a flow rate of 0.4 mL/min. Extracted ion chromatograms were assessed on MassLynx 4.2 for correct analyte peak shape, retention time, and manually integrated when needed.

Supplemental TABLE S2
LC-MS/MS Parameters

| Tandem Mass | Waters Xevo TQ-XS | | | | |
|---|---|---|---|---|---|
| Spectrometer | | | | | |
| UPLC | Waters Acquity | | | | |
| Data Acquisition | Waters MassLynx 4.2 | | | | |
| Software | | | | | |
| MS Source | Electrospray, positive ion mode | | | | |
| Column | Phenomenex Prodigy ODS (3) 100 Å 3 μm 2.0 × 100 mm | | | | |
| Column Temperature | 30° C. | | | | |
| Mobile Phase A | 0.1% Formic Acid in Milli-Q Water | | | | |
| Mobile Phase B | 0.1% Formic Acid in Acetonitrile | | | | |
| | | | | Flow Rate, | |
| Gradient Profile | Time, min | | % B | mL/min | Curve |
| | 0 | | 10 | 0.3 | — |
| | 1 | | 10 | 0.3 | 6 |
| | 5 | | 75 | 0.3 | 6 |
| | 5.01 | | 75 | 0.4 | 6 |
| | 8 | | 75 | 0.4 | 6 |
| | 8.01 | | 10 | 0.4 | 6 |
| | 10 | | 10 | 0.3 | 6 |
| Injection Volume | 15 μL | | | | |
| Capillary | 1 kV | | | | |
| Source Temperature | 110° C. | | | | |
| Desolvation, nebulizer | Nitrogen @ 1,000 L/hr and 300° C. | | | | |
| gas | | | | | |
| Collision gas | Argon @ 0.15 mL/min | | | | |
| Mass Resolution | Unit in both quadrupoles | | | | |
| Run Time | Approximately 10 minutes | | | | |
| | | Precursor | | Product Mass | Collision |
| Monitored Ions | Compound | Mass (Da) | Cone (V) | (Da) | (eV) |
| | Sarin | 141 | 20 | 99 | 10 |
| | IMPA | 139 | 14 | 79 | 20 |
| | | | | 97 | 8 |

A sarin working solution was used to prepare a sarin stock solution in Milli-Q water. A 1000 μg/mL standard of IMPA in methanol was purchased from Cerilliant (Round Rock, Tex., USA). A working stock solution of sarin and IMPA was prepared from the stock solutions in Milli-Q water for use in preparation of calibration standards and quality control (QC) samples. A calibration (standard) curve was analyzed at concentrations of 0.1, 0.5, 1.0, 5.0, 50, 100, 500 and 1,000 ng/mL, with the exception of sarin which had a limit of quantitation (LOQ) of 0.5 ng/mL, not 0.1 ng/mL. A weighted linear regression curve using 1/x² as the weighting factor, with x being the concentration of sarin/IMPA spiked in ng/mL, and y being the sarin/IMPA peak area, was used to calculate the correlation coefficient (r=0.996). The formula for linear regression was used as follows: y=x²+mx+b

Molecular Dynamics and Docking Simulations.

Density functional theory (DFT) calculations. A non-standard residue calculation was performed for the inhibited form of the phosphylated serine after inhibition with sarin. Geometry optimization was done with the hybrid exchange functional and the Lee-Yang-Parr correlation functional (B3LYP)²⁹⁻³¹ in combination with the cc-PVTZ basis set³² and implicit solvation for water using the IEF-PCM solvation method³³⁻³⁵. At the same level of theory, Merz-Kollman electrostatic potential (ESP) calculations were performed to determine the atomic charges of all atoms. The B3LYP/cc-PVTZ method correlates with the charge calculations of the standard residues in the AMBER ff03 force field^{36; 37}.

Protein preparation. For the native enzyme, a crystal structure of human BChE (PDB: 1P0I)⁹ was the starting point. Missing residues, including D378, D379, and N455, were added according to the FASTA sequence and implemented manually using MODELLER³⁸. Missing hydrogens were added using AMBER's xleap^{39; 40} module. The protonation states of the titratable residues were determined using the PDB: 2pqr⁴¹ utility for a pH of 7.4 and several chloride anions were added to neutralize the system. For the inhibited form of BChE by sarin, three different starting points were used. A forward-engineered inhibited structure was created from the native crystal structure of BChE (PDB: 1P0I)⁹. The native structure was edited in UCSF Chimera⁴² to reflect the inhibited form of sarin and missing hydrogens were added with AMBER's xleap^{39; 40} module. Two other preparations of the inhibited structure were done with a crystal structure of human BChE inhibited by enantiopure isomers of (R)-VX (PDB: 2XQJ) and of (S)-VX (PDB: 2XQK)⁴³. Two missing residues at the N-terminus were omitted in the protein preparation from the VX-inhibited enantiomers. The original protein structures were edited to reflect the inhibited form of sarin at the active site serine residue. The partial atomic charges for the atoms for the phosphorylated inhibited of the catalytic serine residue were calculated using the RESP protocol implemented in the Antechamber¹⁸ module in the AMBER 18 package after the previously described ESP calculations. The protonation states of the titratable residues were determined using the pdb2pqr utility for a pH of 7.4⁴¹. All missing hydrogen atoms were added and several chlorides were added to neutralize the system.

For the protein preparations described above, the same was done for the porcinated form of BChE. The mutations for the Y282N/G283H/T284M/P285L native and inhibited forms were made using the most probable Drunback rotamer⁴⁴ as predicted by UCSF Chimera⁴². At the site of the G283H mutation, three possible different protonation states of ε (HIE), δ (HID), and doubly protonated (HIP) histidine were considered.

Molecular dynamics (MD) simulations. All MD simulations were performed using the AMBER ff03 force field^{36; 37} in the presence of explicit water molecules, using a TIP3P⁴⁵ representation. The structures were solvated in an octahedral water box and water molecules were added until about 12 Å away from the enzyme. The MD protocol involved a two-step minimization, a heating step, an equilibration step, and finally a production MD simulation. In the minimization procedure, the solvent and ions were first minimized followed by the entire system for 2500 steps. Before starting the equilibration and production MD simulations, the temperature was raised from 0 to 300 K with a small force constant on the enzyme to resist any drastic changes. The system was then subjected to a 40 ns equilibration step. Normally equilibration steps are on the ˜10 ps time scale, but in order to ensure a constant backbone root mean square deviation (RMSD), especially for the inhibited forms, a longer equilibration was used. Finally, each system was then subjected to a production MD simulation for 50 ns. The number of atoms, temperature, and pressure were kept constant for these NPT MD simulations, and periodic boundary conditions were used. During the production simulation, the time step was set to 2 fs over a 50 ns production step resulting in 25,000 frames for analysis. All distances and RMSD values were measured using AMBER's CPPTRAJ and PYTRAJ modules⁴⁶.

Clustering Analysis. To decrease the amount of structures for analysis, a clustering procedure for each frame was implemented using AMBER's CPPTRAJ module⁴⁶. Similar RMSD values of the protein backbone were grouped in several clusters and a centroid was chosen as a “representative” structure for each cluster. In order to get an accurate representation of all of the possible conformations of the enzyme, five structures were chosen for analysis that represent close to 100% of the 50 ns MD simulation. In the supporting information, RMSD plots are included in order to illustrate several clusters and how they were grouped by similar RMSD values.

Results

Sequence Alignment

To ascertain the root cause of the surprising results with the porcine BChE (McGarry et al 2019, in review), a sequence alignment was conducted comparing Sus scrofa BChE with that of BChE from several primates. While it is expected that multiple amino acids between species would differ, a stretch of four amino acids at positions 282-285, when compared with human, were different from an otherwise conserved region of the enzyme. Two of these residues (284 & 285) are located within the aminoacyl binding pocket of the enzyme, while the other two residues are immediately adjacent to the pocket. While other differences were observed, those differences were not explored for their importance in reactivity at this time.

Purification of Recombinant BChEs

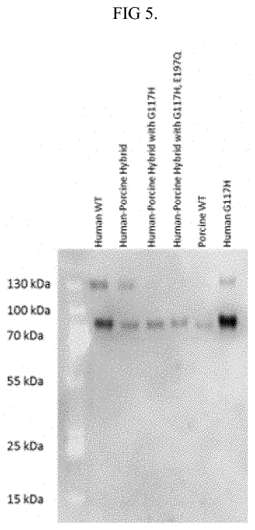

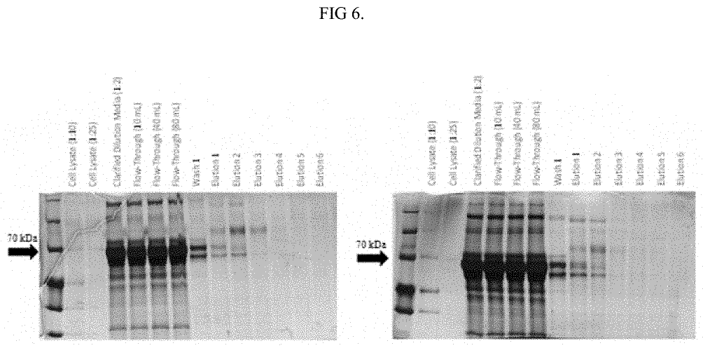

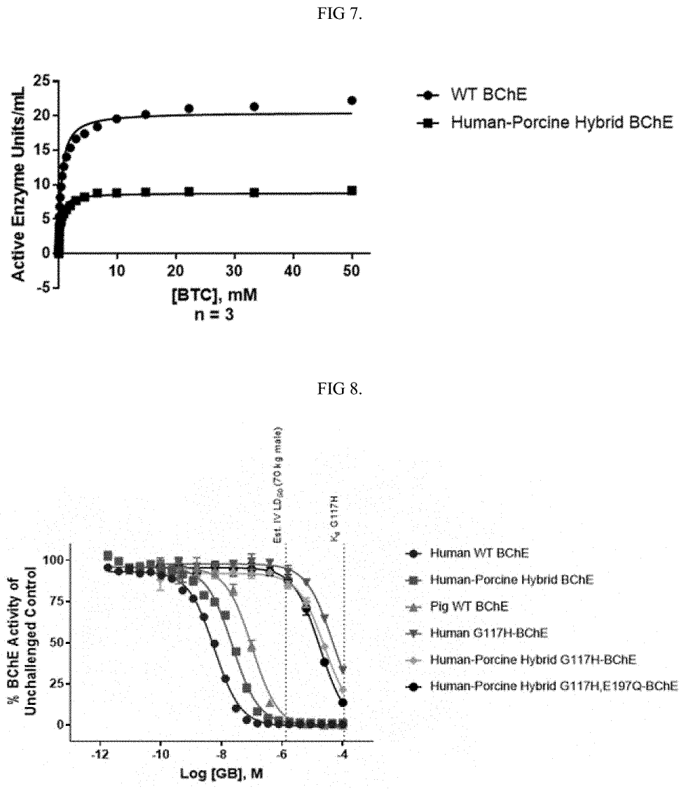

As described above, multiple recombinant BChEs were transiently expressed and purified from HEK293E cells. To assess purity and to ensure the presence of the proteins of interest, silver staining of the purifications of two of the constructs as well as an anti-histidine Western blot of each construct were performed under denaturing conditions. As observed in FIGS. 5 and 6, the histidine-tagged BChEs are clearly present as a prominent band at ˜85 kDa—indicative of the expected size of the fully glycosylated human BChE. The identity of the copurifying, ˜130 kDa, anti-histidine reactive protein in the human WT, human-porcine hybrid, and human G117H variants (FIG. 5) was not determined. An anti-BChE western blot was also performed against the WT and human-porcine hybrid enzymes and confirmed the presence of the BChE (data not shown); however, the antibody that was used appears to have lower sensitivity to the human-porcine hybrid enzyme. Although purification using procainamide resin was attempted, it yielded varying levels of success depending upon the construct; therefore, immobilized metal affinity chromatography (IMAC) purification was used for the purification of all of the constructs discussed herein.

After observing the results discussed above and taking into consideration that fraction 2 yielded the greatest enzymatic activity following preliminary activity analysis, fraction 2 was used for all subsequent experiments, despite the observation of impurities in the silver stain gel (FIG. 6).

To ensure that the purified WT and human-porcine hybrid enzymes catalyzed cleavage of the substrate butyrylthiocholine (BTC) in a similar manner, a substrate titration experiment was performed. As seen in FIG. 7 while both the KM (WT: 456 μM vs. hybrid: 292 μM) and the Vmax (WT: 20.5 vs. hybrid: 8.78), of the hybrid enzyme are lower than that of the WT, the ability to catalyze BTC hydrolysis is not greatly reduced.

Inhibition of Recombinant BChEs by Sarin

Each recombinant enzyme was challenged with varying concentrations of sarin and then activity was assessed. As observed in FIG. 8, similar levels of inhibition are observed in two “clusters” of enzymes. The human WT, porcine WT, and human-porcine hybrid BChE all display similar IC50 values (5.8 nM, 23.0 nM, and 103.2 nM, respectively). Additionally, as controls, and in an attempt to improve the human-porcine hybrid BChE, variations of BChE were created using mutations that were previously identified and characterized in depth 12; 16; 17; 20; 47. Interestingly, each of these enzymes (human G117H, human-porcine hybrid with G117H, and human-porcine hybrid with G117H, E197Q) cluster together with a much higher IC50 than that of the other three enzymes (45.6 μM, 24.3 μM and 17.7 μM, respectively). High affinity for the substrate of interest is a desirable component for all candidate therapeutic bioscavengers, and included in this figure as a point of reference are the reported Kd for the G117H mutation 20 as well as the estimated intravenous dose corresponding to 50% lethality (IV LD50) of sarin for a 70 kg human male 48. It should be noted that the estimated IV LD50 was converted to the corresponding molar concentration in the blood based on the published results using the estimated blood volume of a 70 kg human male to be 5.250 L. The kcat and Kd/Km has been a point of emphasis for years regarding the G117H mutation's inability to efficiently catalyze the hydrolysis of nerve agents. As shown below, the driving property behind the ineffectiveness of G117H likely stems from a low affinity of the nerve agent for this enzyme. On the contrary, human WT BChE (which is currently in clinical trials as a stoichiometric bioscavenger), the porcine WT BChE, and the human-porcine hybrid BChE enzymes all display much higher affinity for sarin and are readily inhibited by the agent. Furthermore, the rate of inhibition (ki) of each of the recombinant enzymes are similar (Table 1; n=6). Additionally, the recombinant enzymes, with the exception of recombinant human WT BChE (which is an order of magnitude lower), display a comparable ki to that of the plasma counterparts as described and reproduced here.

TABLE 1
ki Calculations for the Inhibition of WT and Recombinant BChEs by Sarin

| | | | | | | Recombinant |
|---|---|---|---|---|---|---|
| | | | | | | Human- |
| | | Gottingen | Yorkshire | Recombinant | Recombinant | Porcine |
| | Human | Mini Pig | Swine | Human | Pig WT | Hybrid |
| | (Plasma) | (Plasma) | (Plasma) | WT BChE | BChE | BChE |
| Mean kᵢ | 8.30 ± 1.41 × 10⁶ | 0.63 ± 0.11 × 10⁶ | 1.36 ± 0.19 × 10⁶ | 0.45 ± 0.03 × 10⁶ | 0.97 ± 0.17 × 10⁶ | 2.31 ± 0.43 × 10⁶ |
| (M⁻¹min⁻¹) | | | | | | |

Aging and Spontaneous Reactivation of Recombinant BChEs

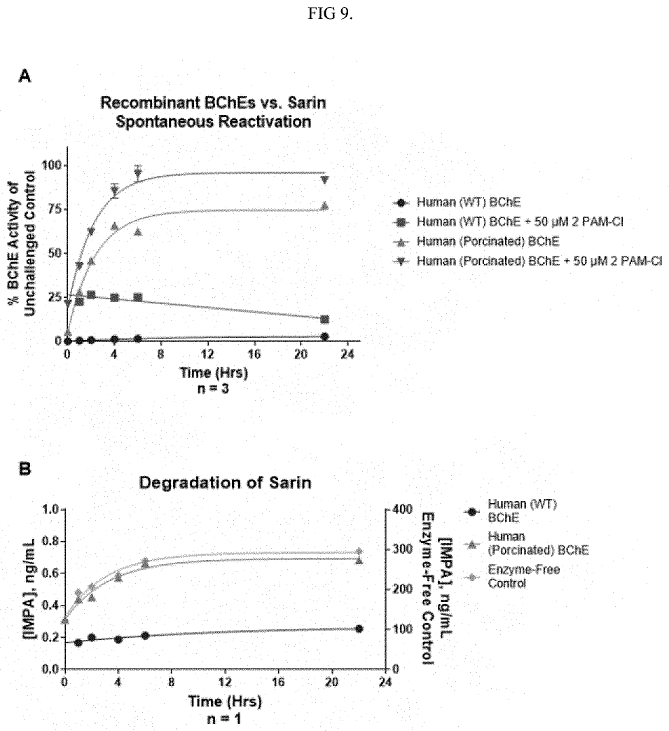

As observed in FIG. 9, panel A, the human-porcine hybrid BChE displays spontaneous reactivation with a t1/2 of 2.4 hours and a maximal reactivation of approximately 75% of the activity of the unchallenged control. The addition of 2-PAM Cl results in an additional 15-30% activity. These experiments were also carried out in multiple, separate experiments to 48 hours with similar results. The 22-hour experiment is shown below as the time points utilized for this experiment were also analyzed by mass spectrometry—an analysis that was not performed in the 48-hour experiments.

FIG. 9, panel B (and Supplemental Table S3) shows the measured concentrations of IMPA (ng/mL), the postulated primary degradation product of sarin, in both the human wild type and human-porcine hybrid BChE enzymes that were challenged with sarin. The samples that were frozen at −70° C. were thawed and aliquoted for analysis without any additional sample manipulation. The reported concentrations are in ng/mL and were calculated using an eight-point standard calibration curve that went from 0.1 to 1,000 ng/mL. The targeted LC-MS/MS method that was used monitored the IMPA 139>97 ion transition and the determined concentrations are plotted (FIG. 9, panel B and Supplemental Table S3). The concentrations of IMPA increased from 0.31 to 0.66 ng/mL in the first 8 hours for the human-porcine hybrid BChE, indicative of the degradation of sarin. In the human WT BChE, an increase in IMPA was observed, but to a much lower extent, from 0.17 to 0.21 ng/mL in the first 8 hours. The LC-MS/MS also monitored for the presence of sarin in these samples. The detection limit for sarin was 0.5 ng/mL and, as expected, it was not detected at any of the timepoints because all sarin is presumably covalently bound to the protein. Additionally, included in FIG. 9, panel B, a plot of the sarin-only control in buffer alone is plotted on the right-hand axis to observe the natural hydrolysis reaction over time (the concentration of IMPA increased from 122 to 295 ng/mL in this case). Much greater IMPA concentrations were observed from the enzyme-free controls due to the higher initial, unbound concentrations.

Supplemental TABLE S3
Measured Concentrations of Sarin and IMPA using LC-MS/MS

| | Time | [Sarin] | [IMPA] |
|---|---|---|---|
| Sample ID | (hours) | (ng/mL) | (ng/mL) |
| Human (WT) BChE | 0 | ND | ND |
| Human (WT) BChE | 1 | ND | 0.17 |
| Human (WT) BChE | 2 | ND | 0.20 |
| Human (WT) BChE | 4 | ND | 0.19 |
| Human (WT) BChE | 6 | ND | 0.21 |
| Human (WT) BChE | 22 | ND | 0.26 |
| Human (Porcinated) | 0 | ND | 0.31 |
| BChE | | | |
| Human (Porcinated) | 1 | ND | 0.44 |
| BChE | | | |
| Human (Porcinated) | 2 | ND | 0.45 |
| BChE | | | |
| Human (Porcinated) | 4 | ND | 0.57 |
| BChE | | | |
| Human (Porcinated) | 6 | ND | 0.66 |
| BChE | | | |
| Human (Porcinated) | 22 | ND | 0.68 |
| BChE | | | |

(ND = Not Detected)

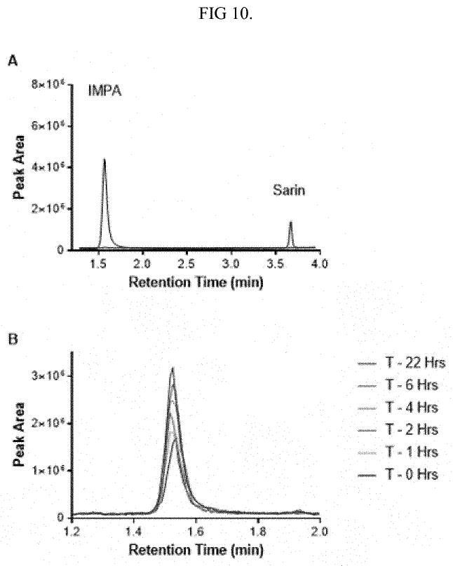

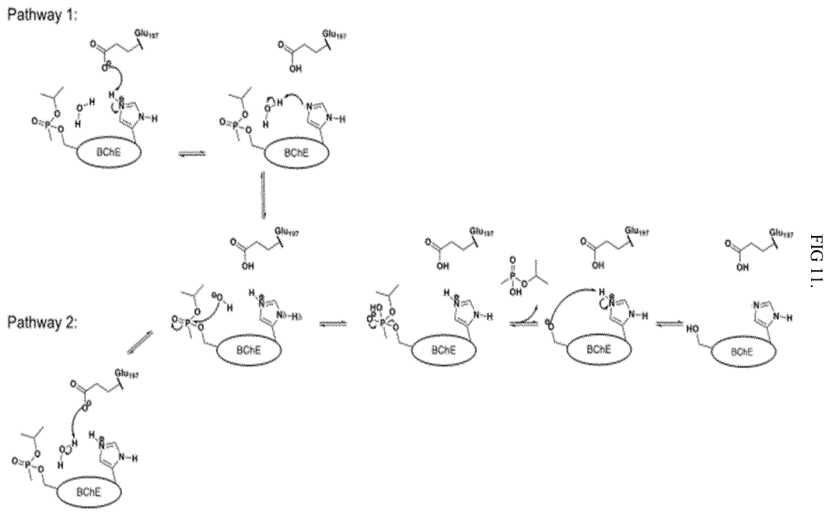

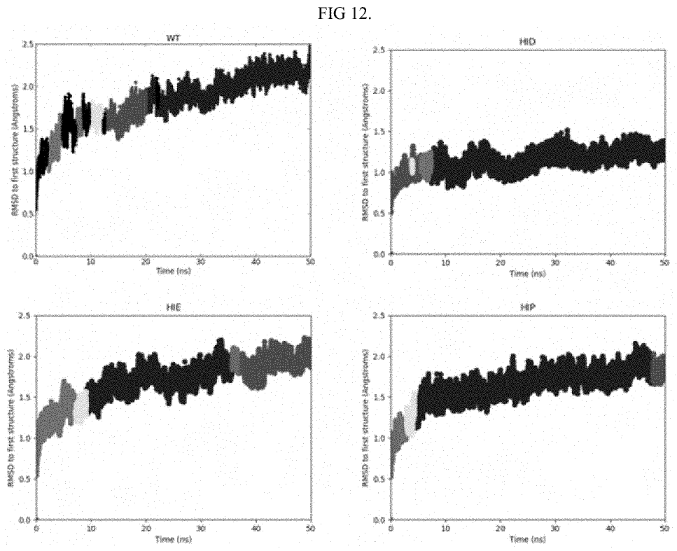

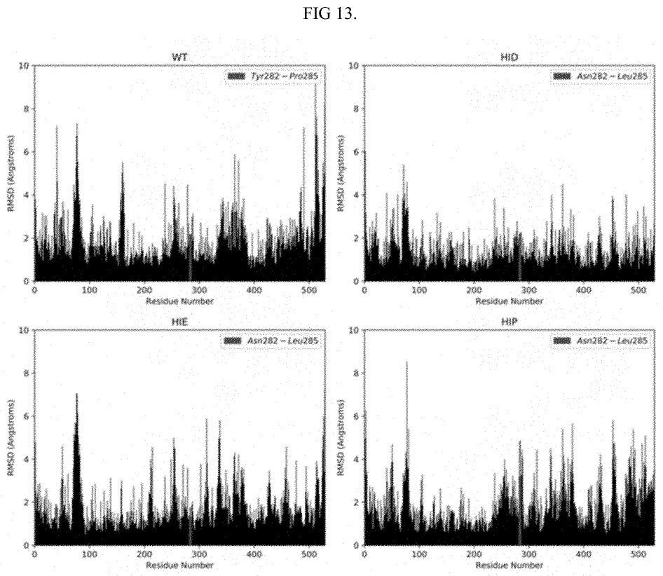

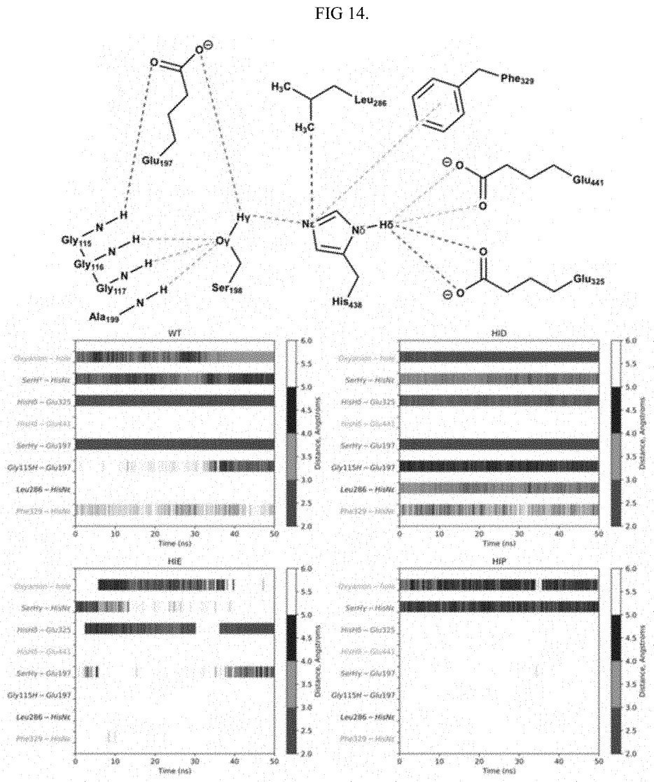

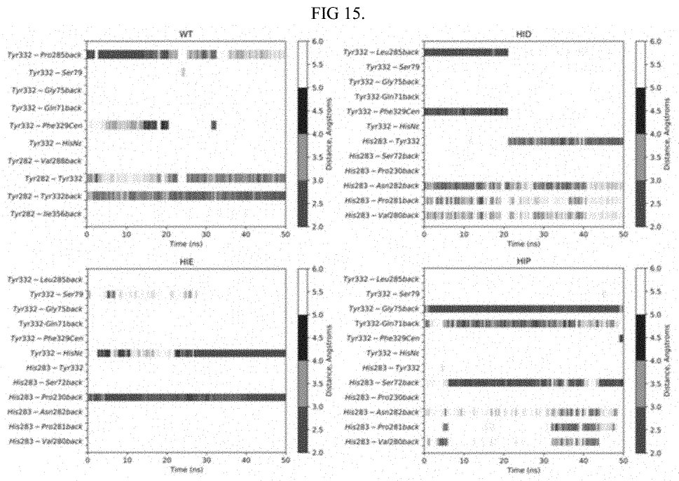

An overlay of the extracted ion chromatograms for a 10 ng/mL solvent standard of IMPA (137>97) and sarin (141>99) can be seen in FIG. 10, panel A. The observed retention times for the two ions were 1.56 min for IMPA and 3.66 min for sarin. FIG. 10, panel B shows a magnified region of the chromatogram (1.2-2.0 min) with all of the timepoints plotted for the human-porcine hybrid BChE samples. The IMPA peak areas (˜1.56 min) increase with each timepoint, indicating the degradation of sarin over time.

In addition to the LC-MS/MS data, GC-HRMS data obtained from dilutions following a 500-fold molar excess challenge indicated a slight difference in the kinetic profile of the degradation of sarin over time in the presence of the porcinated-human hybrid enzyme when compared to the no enzyme control (see Supplemental Table S4). It should be noted that unlike the aforementioned aging experiments, excess, unbound agent was not removed using spin columns in this experiment. As such, the corresponding activity results indicated that in the presence of 500-fold molar excess sarin, the hybrid BChE was effectively rendered inactive for the duration of the experiment with enzymatic activity only increasing by 1% over the 5-hour timeframe. Following a challenge using a 10-fold molar excess of sarin, enzymatic activity was beginning to recover, albeit slowly (3.5% total increase, 13.3% at 2 hours to 16.8% at 5 hours post-challenge, data not shown).

Supplemental TABLE S4
Measured Concentrations of sarin overtime using GC-HRMS

| | Time | [Sarin] |
|---|---|---|
| Sample ID | (hours) | (ng/mL) |
| 10 mM MOPS (—) Control | 0 | 1.61 |
| 10 mM MOPS (—) Control | 2 | 0.84 |
| 10 mM MOPS (—) Control | 5 | 0.60 |
| Porcinated-BChE | 0 | 1.57 |
| Porcinated -BChE | 2 | 0.97 |
| Porcinated -BChE | 5 | 0.42 |

Molecular dynamics (MD) simulations of WT and hybrid BChE before and after inhibition with sarin

As to gain a better understanding for the potential of active WT and potential for enzyme reactivation, computational tools were employed. The WT enzyme was evaluated for a 50 ns MD simulation in order to understand the important interactions of the native and inhibited forms. The Y282N/G283H/T284M/P285L variant was also evaluated for 50 ns, taking into consideration three different possible protonation states of ε (HIE), δ (HID), and double protonated (HIP) histidine at the site of the G283H mutation.

The active site distances of the WT enzyme and the three different mutant structures were evaluated. Active site distances that were evaluated for the native forms of the enzyme include the Oy of Ser198 and the hydrogens from the backbone amides of Gly116, Gly117, and Ala199 (oxyanion hole), the interactions of Hγ of Ser198 with the NE of His438 (SerHγ-HisNε) and oxyanion of Glu197 (SerHγ-Glu197), the Hδ interactions of His438 (Glu325 and Glu441), the interaction of the backbone amide to Glu197, and the measurement of the proximity of the acyl-pocket residues Leu286 and Phe329 to the active site (Leu286-HisNε and Phe329-HisNε). To evaluate the impact at the site of the mutation, important interaction distances of Tyr282, Pro285, and Tyr332 were measured for the WT enzyme and distances of His283, Leu285, and Tyr332 were measured for the mutated enzyme.

The active site distances of the WT and HID forms of the native enzyme were observed to be quite similar; however, the oxyanion hole created by the Oy of Ser198 and the hydrogens from the backbone amides of Gly116, Gly117, and Ala199 are much shorter in the HID form. The decrease in the distance of the oxyanion hole is possibly as a result of the encroachment of Leu286 and Phe329 into the active site (shown in FIG. 17). Leu 286 is adjacent to the 4-residue mutation, and the observed encroachment may be a result of the P285L mutation. Tyr282 and the backbone of Pro285 of the WT form seems to “anchor” the position of the α-helix that includes several important residues, such as Tyr332, Phe329, and Glu325. Leu285 seems to “push” the α-helix and allows Leu286 to move into the active site. In the WT form of the enzyme, the α-helix containing Tyr332, Phe329, and Glu325 is situated very close to the acyl loop. The HID form of the His283 mutation is situated above the acyl-loop and the 6-hydrogen shows a small interaction with the alcohol group of Tyr332 as it is pushed away from interaction with backbone of Leu285.

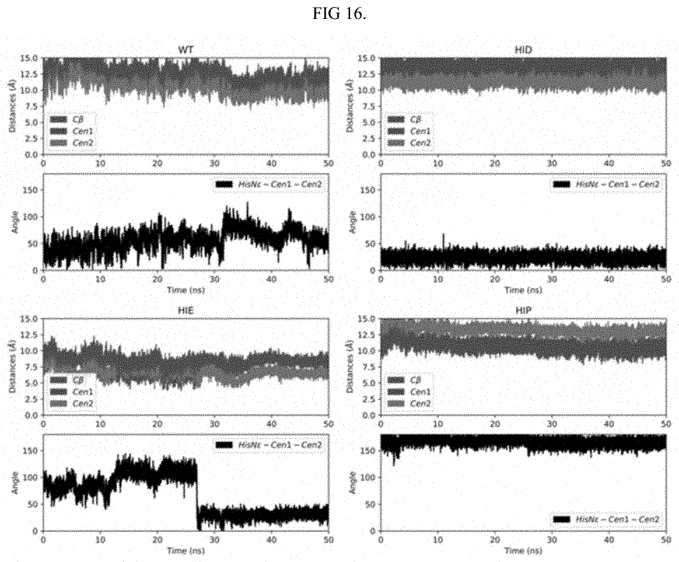

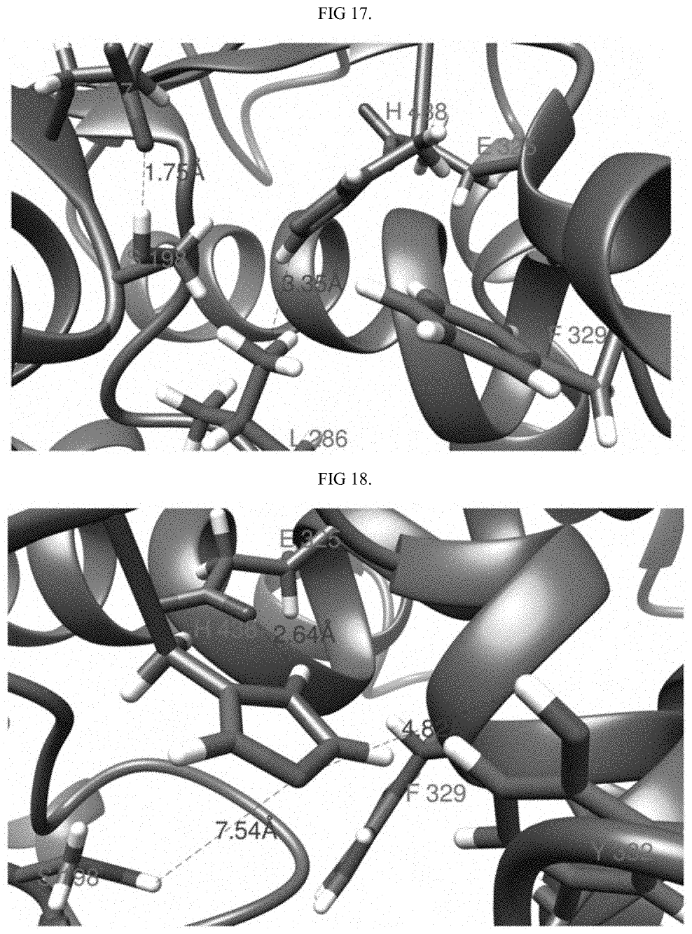

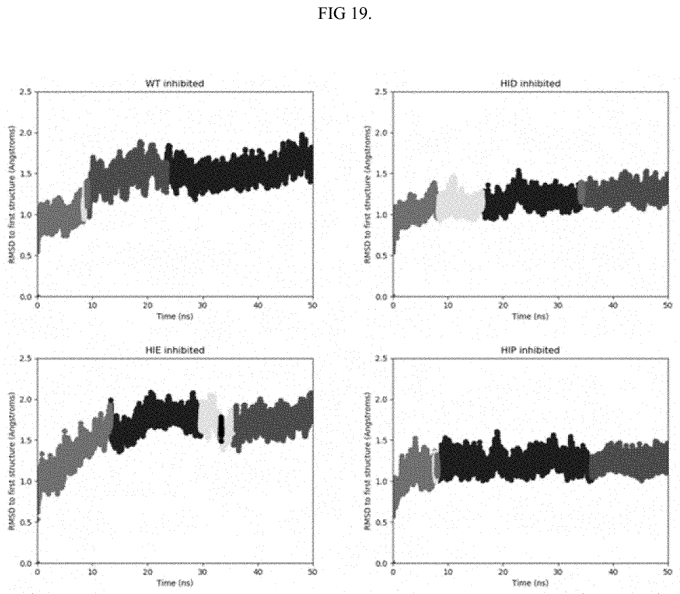

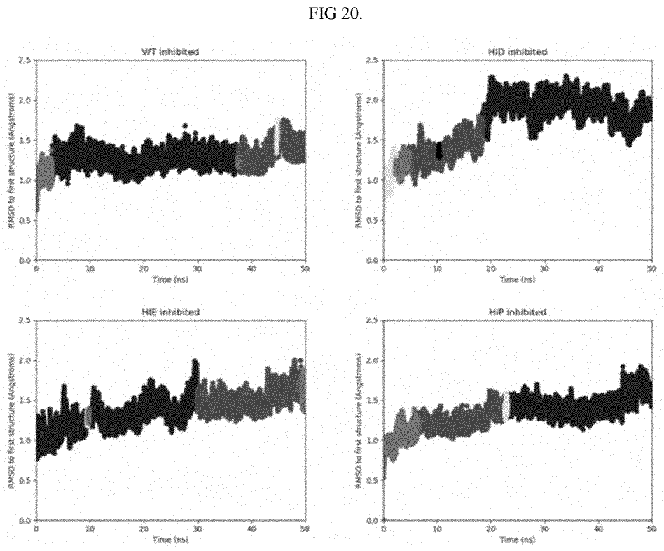

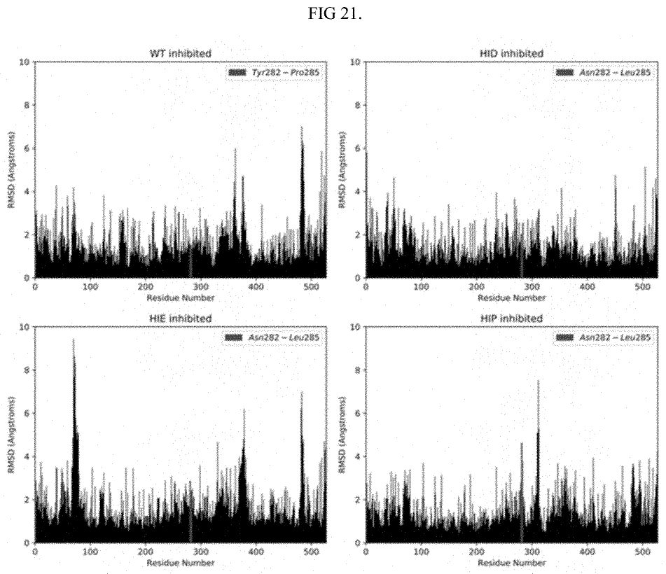

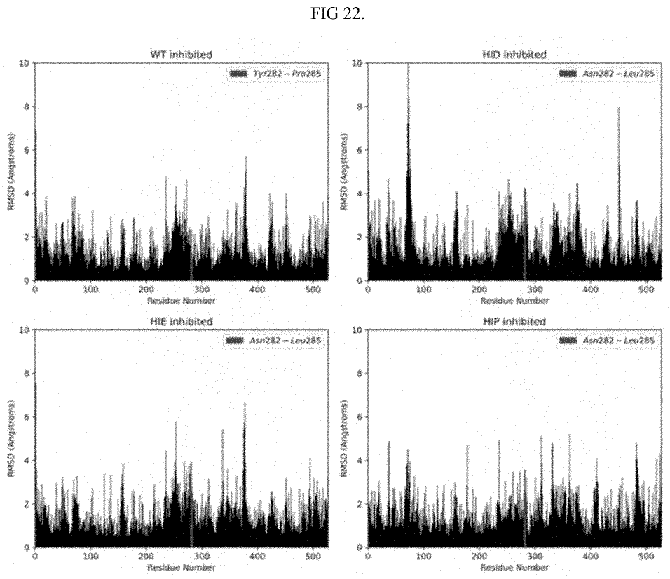

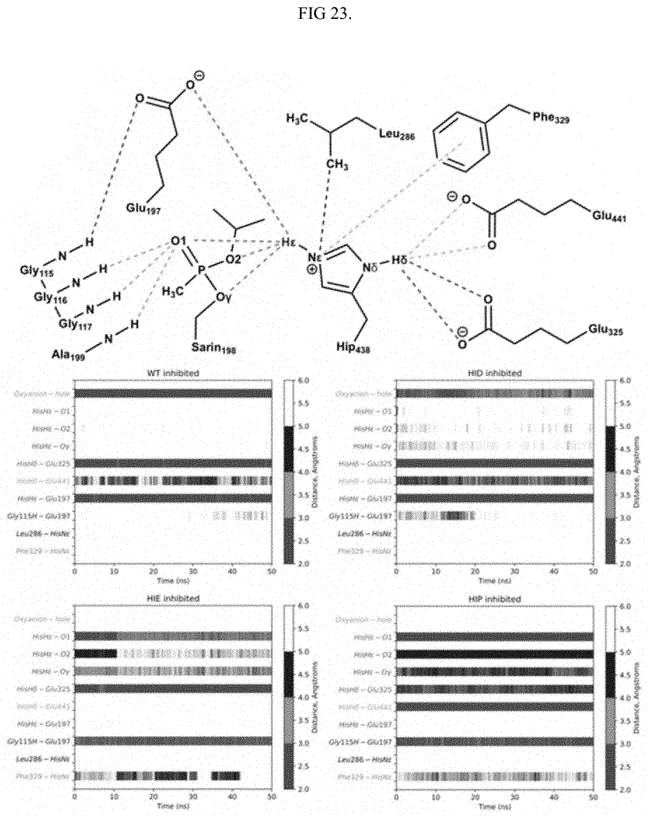

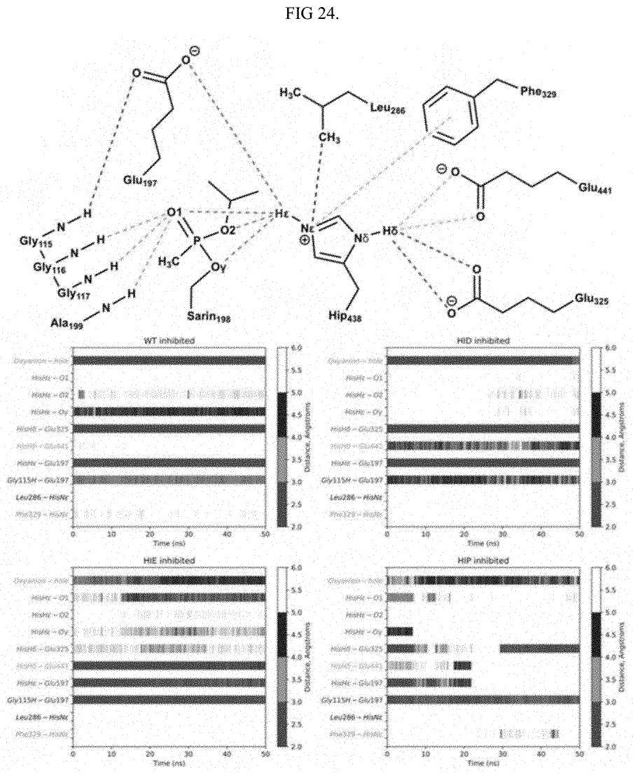

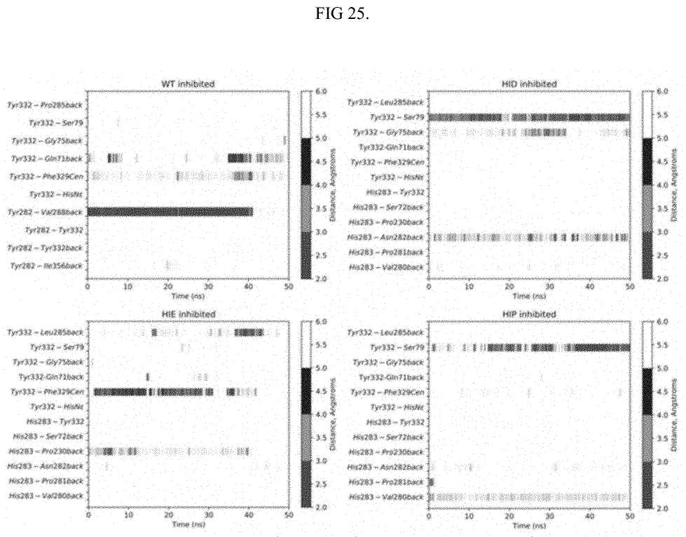

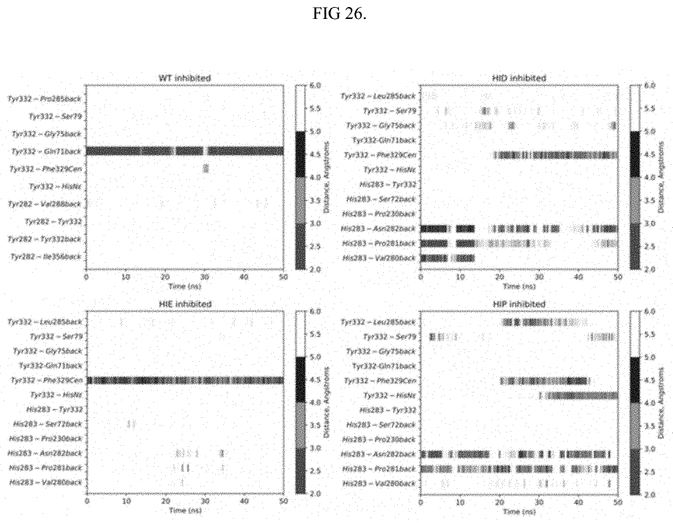

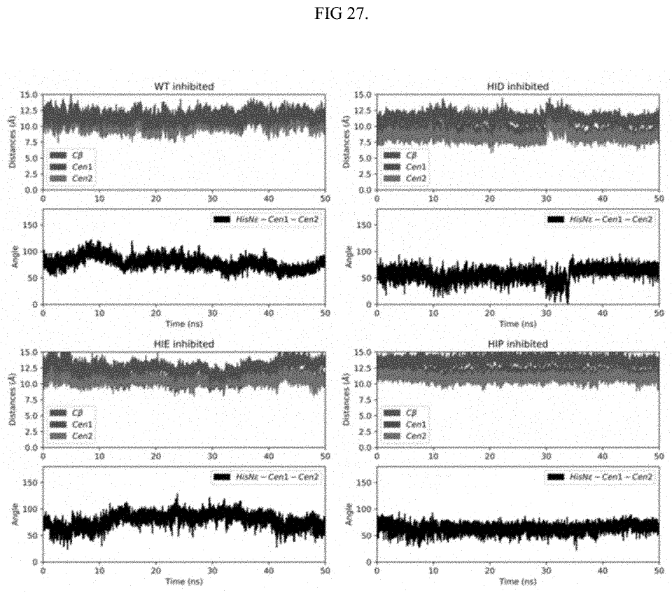

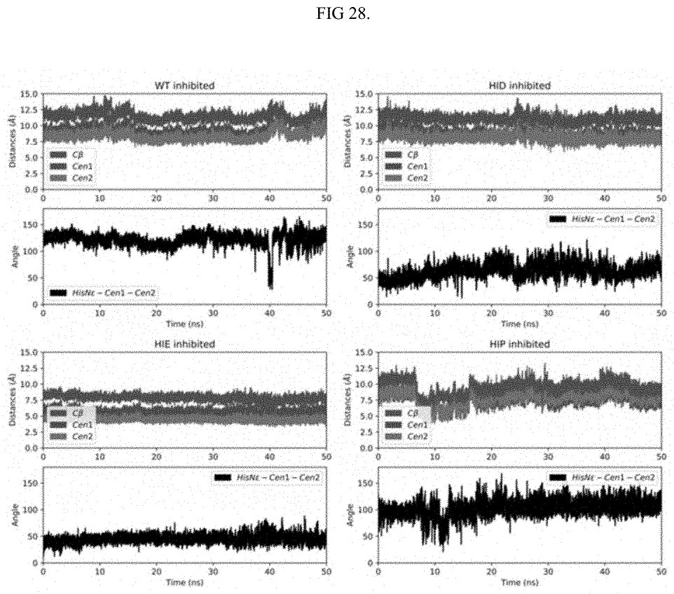

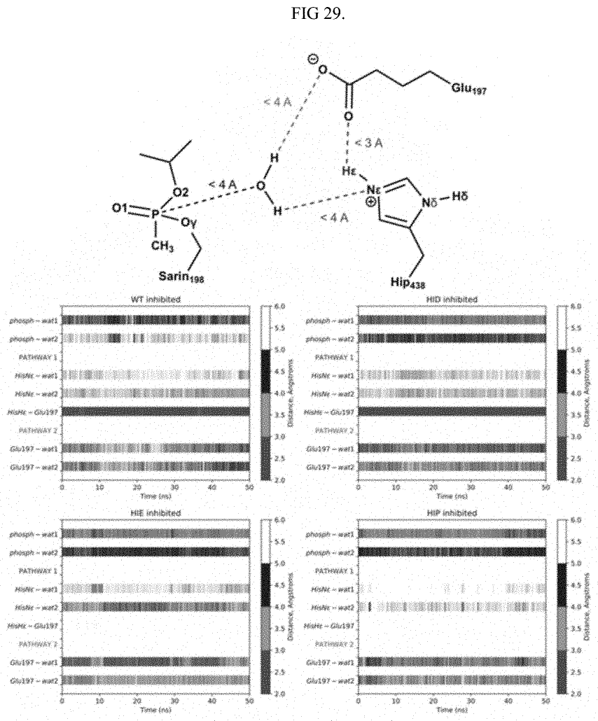

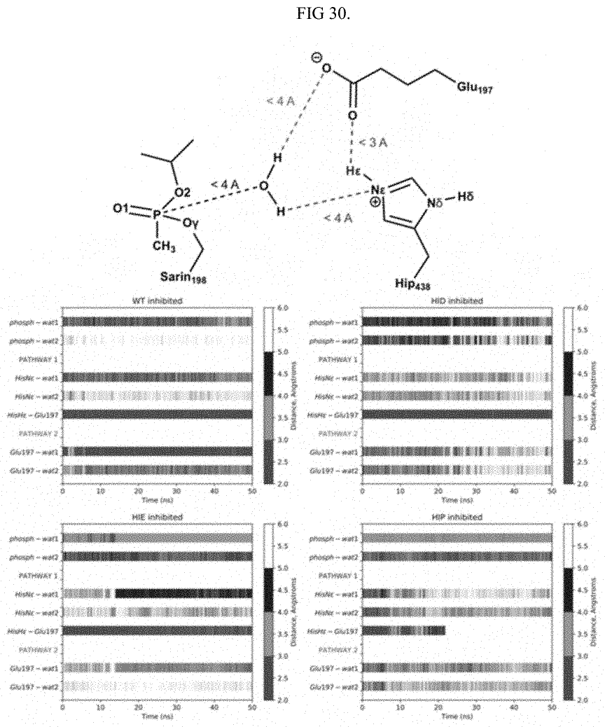

The active site interactions of the HIE and HIP forms of the His283 hybrid enzyme were quite different from the WT enzyme. Noticeable differences were observed in the lack of interaction between the Hγ of Ser198 and the oxyanion of Glu197 as well as the weak interaction of the Hγ of Ser198 and HisNε. In the HIE form of the His283 mutation, the catalytic His438 moves away from the active site and into the active site gorge where there is little to no interaction with the Hγ of Ser198. As a result, for the hybrid enzyme, the catalytic triad transforms into a catalytic dyad. The active site climb can also be observed by the very close distance of ˜7.5 angstroms to the Trp82 residue of the Ω-loop (shown in FIG. 16). As a result of the HIE mutation, there is a very strong interaction between the ε-hydrogen of His283 and the carbonyl backbones of Pro230 and Trp231 siting at the bottom of the acyl-binding pocket. Tyr332 lacks the same strong interaction with the Pro285 backbone and enters the active site to interact with the catalytic His438 after climbing into the active site gorge. The HIP form of the native His438 enzyme performs a similar “climbing” of the active site, except not nearly as high of a climb, as evidence by the Trp82 distances sitting at ˜10 Å. Instead, the catalytic His438 sits just above Ser198 and forms a catalytic dyad by a strong hydrogen bonding interaction of Ho of His438 and Glu197.

The observed differences in the native enzyme from WT to mutated forms suggests a possible explanation for the decrease in vₘₐₓ for the hybrid enzyme.

The inhibited forms of the BChE enzyme were studied with two different starting PDB structures (2XQJ and 2XQK), which are inhibited forms of the less and more toxic stereoisomers of VX, respectively. To evaluate each active site, similar distances to the native form were measured; however, the distances to the Oy of phosphylated Ser198 (Sarin198) has been split into 4 distances in order to emphasize the different distances of the phosphorus oxygens near the oxyanion hole (Oxyanion-hole and HisHε-O1), the bridging oxygen to the isopropyl leaving group (HisHε-O2) and the oxygen of the original Ser198 group (HisHε-Oγ). As seen in the native form, the active sites are quite similar for the 2XQJ simulations between the WT inhibited and the hybrid HID inhibited forms. A small difference is the decrease of interaction between the oxyanion hole in the HID form, but one does observe a very weak to no interaction of ˜5 Å to the HisHε. For 2XQJ, the HIE and HIP forms of the mutation lack the strong oxyanion hole interactions of the WT and HID forms and as a result, the phosphorus oxygens show a much closer interaction to HisHε. In 2XQJ, the movement of the phosphylated Ser198 to His438 allows for a very strong interaction of the Gly115 amide hydrogen backbone to the oxyanion of Glu197 that is maintained throughout the simulation. However, the HID and HIP forms of the inhibited enzyme lack the very strong HisHε-Glu197 interactions at His438 that are observed in the HID and WT forms of the inhibited enzyme. In the inhibited forms of 2XQK, the WT and HID forms show similar interactions, but the phosphorus-oxygen distances are now closer for HisHε in the WT form, rather than the HID form. The WT form of 2XQK also show mild to strong interactions for the amide hydrogen backbone of Gly115H to the oxyanion of Glu197.

Distance Analysis for the Active Site of the Native Enzyme

To observe the important interactions in the active site of the WT and hybrid forms of the native enzyme, AMBER's PYTRAJ module was used in order to extract distances. Heat map plots were made for the WT and the HID, HIE, and HIP protonation states of the G283H mutation were made. The heat maps display the distances as a function of color over time. The interatomic distances measured are summarized below:

Interatomic Distances:

Oxyanion hole: Average distance from hydrogens from the backbone amides of Gly116, Gly117, and Ala199 to the Oγ of Ser198.

SerHγ-HisNε: Distance from the Hγ of Ser198 to the ε-nitrogen of the catalytic His438.

HisHδ-Glu325: Distance from the δ-hydrogen of the catalytic His438 to the catalytic Glu325 oxyanion.

HisHδ-Glu441: Distance from the δ-hydrogen of the catalytic His438 to the non-catalytic Glu441 oxyanion, this is an interaction that is often observed in the inhibited form of the enzyme.

SerHγ-Glu197: Distance from the Hγ of Ser198 to the adjacent Glu197 oxyanion.

Gly115H-Glu197: Distance from the hydrogen from the backbone amide of Gly115 to the Glu325 oxyanion.

Leu286-HisNε: Distance from the closest carbon of the methyl group on Leu286 in the acyl-binding pocket to the ε-Nitrogen of the catalytic His438.

Phe329-HisNε: Distance from the centroid of the benzyl group of Phe329 in the acyl-binding pocket to the ε-Nitrogen of the catalytic His438.

Distance Analysis for the Mutation Site/Acyl Loop of the Native Enzyme

To observe the important interactions in the acyl loop and the site of the mutation of the WT and hybrid forms of the native enzyme, AMBER's PYTRAJ module was used in order to extract distances. Heat map plots were made for the WT and the HID, HIE, and HIP protonation states of the G283H mutation were made. The heat maps display the distances as a function of color over time. The top 6 interatomic distances are shared between the WT and hybrid forms of the enzyme

Shared Interatomic Distances:

Tyr332-Pro285back/Leu285back: Distance from the hydrogen of the alcohol group of Tyr332 to the backbone of Pro285 (WT) or Leu285 (hybrid) backbone.

Tyr332-Ser79: Distance from the Distance from the hydrogen of the alcohol group of Tyr332 to the oxygen of the alcohol group of Ser79 located on the Ω-loop.

Tyr332-Gly75back: Distance from the hydrogen of the alcohol group of Tyr332 to the backbone of Gly75 located on the Ω-loop.

Tyr332-Gln71back: Distance from the hydrogen of the alcohol group of Tyr332 to the backbone of Gln71 located on the Ω-loop.

Tyr332-Phe329Cen: Distance from the hydrogen of the alcohol group of Tyr332 to the centroid of the benzyl group of Phe329 in the acyl-binding pocket.

Tyr332-HisNε: Distance from the hydrogen of the alcohol group of Tyr332 to the ε-nitrogen of the catalytic His438 located in the active site.

WT interatomic distances:

Tyr282-Val288back: Distance from the hydrogen of the alcohol group of Tyr282 to the backbone of Val288 located in the acyl-binding pocket.

Tyr282-Tyr332: Distance from the hydrogen of the alcohol group of Tyr282 to the hydrogen of the alcohol group of Tyr332.

Tyr282-Tyr332back: Distance from the hydrogen of the alcohol group of Tyr282 to the backbone of Tyr332.

Tyr282-Ile356back: Distance from the hydrogen of the alcohol group of Tyr282 to the backbone of Ile356.

Hybrid interatomic distances:

His283-Tyr332: Distance from the δ-(HID) or ε-(HIE) hydrogen or the closest of the two (HIP) of His283 to the oxygen of the alcohol group of Tyr322.

His283-Ser72back: Distance from the δ-(HID) or ε-(HIE) hydrogen or the closest of the two (HIP) of His283 to the backbone of Ser72 located on the Ω-loop.

His283-Pro230back: Distance from the δ-(HID) or ε-(HIE) hydrogen or the closest of the two (HIP) of His283 to the backbone of Pro230 located on the bottom of the acyl-binding pocket.

His283-Asn282back: Distance from the δ-(HID) or ε-(HIE) hydrogen or the closest of the two (HIP) of His283 to the backbone of Asn282 located above the acyl-binding pocket.

His283-Pro281back: Distance from the δ-(HID) or ε-(HIE) hydrogen or the closest of the two (HIP) of His283 to the backbone of Pro281 located above the acyl-binding pocket.

His283-Val280back: Distance from the δ-(HID) or ε-(HIE) hydrogen or the closest of the two (HIP) of His283 to the backbone of Val280 located above the acyl-binding pocket.

Distance Analysis for Trp82 of the Native Enzyme

To visualize the stability and proximity of the omega loop to the active site of the native enzyme, AMBER's PYTRAJ module was used in order to extract distances as well as an angle. Important distances from the ε-Nitrogen of the catalytic His438 to the β-carbon (Cβ), the centroid of the amino ring (Cen1), and the centroid of the benzyl ring (Cen2) of Trp82. A summary of the distances and the angle measured are shown below:

Interatomic Distances:

Cβ: Distance from the ε-nitrogen of the catalytic His438 located in the active site to the β-Carbon of Trp82.

Cen1: Distance from the ε-nitrogen of the catalytic His438 located in the active site to the centroid of the amino ring of Trp82.

Cen2: Distance from the ε-nitrogen of the catalytic His438 located in the active site to the centroid of the benzyl ring of Trp82.

Interatomic Angle:

HisNε-Cen1-Cen2: Angle formed by the ε-nitrogen of the catalytic His438 located in the active site to the centroid of the amino ring of Trp82 to the centroid of the benzyl ring of Trp82.

Distance Analysis for the Active Site of the Inhibited Enzyme

To observe the important interactions in the active site of the WT and hybrid forms of the inhibited enzyme, AMBER's PYTRAJ module was used in order to extract distances. Heat map plots were made for the WT and the HID, HIE, and HIP protonation states of the G283H mutation for 2XQJ and 2XQK were made. The heat maps display the distances as a function of color over time. The interatomic distances measured are summarized below:

Interatomic Distances:

Oxyanion hole: Average distance from hydrogens from the backbone amides of Gly116, Gly117, and Ala199 to the 01 of phosphylated Ser198 (Sarin198) which is the oxygen that is located closest to these backbone amides.

HisHε-O1: Distance from the δ-hydrogen of the catalytic His438 to the O1 of phosphylated Ser198 (Sarin198) which is the oxygen that is located in the oxyanion hole.

HisHε-O2: Distance from the δ-hydrogen of the catalytic His438 to the O2 of phosphylated Ser198 (Sarin198) which is the oxygen connected to the isopropyl leaving group.

HisHε-Oγ: Distance from the ε-hydrogen of the catalytic His438 to the O1 of phosphylated Ser198 (Sarin198) which is the oxygen connected to the protein.

HisHδ-Glu325: Distance from the δ-hydrogen of the catalytic His438 to the catalytic Glu325 oxyanion.

HisHδ-Glu441: Distance from the δ-hydrogen of the catalytic His438 to the non-catalytic Glu441 oxyanion, this is an interaction that is often observed in the inhibited form of the enzyme.

HisHε-Glu197: Distance from the ε-hydrogen of the catalytic His438 to the Glu197 oxyanion.

Gly115H-Glu197: Distance from the hydrogen from the backbone amide of Gly115 to the Glu325 oxyanion.

Leu286-HisNε: Distance from the closest carbon of the methyl group on Leu286 in the acyl-binding pocket to the ε-Nitrogen of the catalytic His438.

Phe329-HisNε: Distance from the centroid of the benzyl group of Phe329 in the acyl-binding pocket to the ε-Nitrogen of the catalytic His438.

Distance Analysis for the Mutation Site/Acyl Loop of the Inhibited Enzyme

To observe the important interactions in the acyl loop and the site of the mutation of the WT and hybrid forms of the inhibited enzyme, AMBER's PYTRAJ module was used in order to extract distances. Heat map plots were made for the WT and the HID, HIE, and HIP protonation states of the G283H mutation for 2XQJ and 2XQK were made. Heat maps display the distances as a function of color over time. The top 6 interatomic distances are shared between the WT and hybrid forms of the enzyme

Shared Interatomic Distances:

Tyr332-Pro285back/Leu285back: Distance from the hydrogen of the alcohol group of Tyr332 to the backbone of Pro285 (WT) or Leu285 (hybrid) backbone.

Tyr332-Ser79: Distance from the Distance from the hydrogen of the alcohol group of Tyr332 to the oxygen of the alcohol group of Ser79 located on the Ω-loop.

Tyr332-Gly75back: Distance from the hydrogen of the alcohol group of Tyr332 to the backbone of Gly75 located on the Ω-loop.

Tyr332-Gln71back: Distance from the hydrogen of the alcohol group of Tyr332 to the backbone of Gln71 located on the Ω-loop.

Tyr332-Phe329Cen: Distance from the hydrogen of the alcohol group of Tyr332 to the centroid of the benzyl group of Phe329 in the acyl-binding pocket.

Tyr332-HisNε: Distance from the hydrogen of the alcohol group of Tyr332 to the ε-nitrogen of the catalytic His438 located in the active site.

WT interatomic distances:

Tyr282-Val288back: Distance from the hydrogen of the alcohol group of Tyr282 to the backbone of Val288 located in the acyl-binding pocket.

Tyr282-Tyr332: Distance from the hydrogen of the alcohol group of Tyr282 to the hydrogen of the alcohol group of Tyr332.

Tyr282-Tyr332back: Distance from the hydrogen of the alcohol group of Tyr282 to the backbone of Tyr332.

Tyr282-Ile356back: Distance from the hydrogen of the alcohol group of Tyr282 to the backbone of Ile356.

Hybrid Interatomic Distances:

His283-Tyr332: Distance from the δ-(HID) or ε-(HIE) hydrogen or the closest of the two (HIP) of His283 to the oxygen of the alcohol group of Tyr322.

His283-Ser72back: Distance from the δ-(HID) or ε-(HIE) hydrogen or the closest of the two (HIP) of His283 to the backbone of Ser72 located on the Ω-loop.

His283-Pro230back: Distance from the δ-(HID) or ε-(HIE) hydrogen or the closest of the two (HIP) of His283 to the backbone of Pro230 located on the bottom of the acyl-binding pocket.

His283-Asn282back: Distance from the δ-(HID) or ε-(HIE) hydrogen or the closest of the two (HIP) of His283 to the backbone of Asn282 located above the acyl-binding pocket.

His283-Pro281back: Distance from the δ-(HID) or ε-(HIE) hydrogen or the closest of the two (HIP) of His283 to the backbone of Pro281 located above the acyl-binding pocket.

His283-Val280back: Distance from the δ-(HID) or ε-(HIE) hydrogen or the closest of the two (HIP) of His283 to the backbone of Val280 located above the acyl-binding pocket.

Distance Analysis for Spontaneous Hydrolysis of the Inhibited Enzyme

To observe the important water interactions in the WT and hybrid forms of the inhibited enzyme, AMBER's PYTRAJ module was used in order to extract distances. Heat map plots were made for the WT and the HID, HIE, and HIP protonation states of the G283H mutation for 2XQJ and 2XQK were made. The heat maps display the distances as a function of color over time. The interatomic distances measured are summarized below:

Interatomic Distances:

phosph-wat1: Distance from the phosphorus of the inhibited Ser198 to the oxygen of the closest water to the phosphorous atom of the inhibited Ser198.

phosph-wat2: Distance from the phosphorus of the inhibited Ser198 to the oxygen of the 2^{nd} closest water to the phosphorous atom of the inhibited Ser198.

HisNε-wat1: Distance from the ε-nitrogen of the catalytic His438 to the center of mass of the hydrogens of the closest water to the phosphorus atom of the inhibited Ser198.

HisNε-wat2: Distance from the ε-nitrogen of the catalytic His438 to the center of mass of the hydrogens of the 2^{nd} closest water to the phosphorus atom of the inhibited Ser198.

HisHε-Glu197: Distance from the ε-hydrogen of the catalytic His438 to the Glu197 oxyanion.

Glu197-wat1: Distance from the Glu197 oxyanion to the center of mass of the hydrogens of the closest water to the phosphorus atom of the inhibited Ser198.

Glu197-wat2: Distance from the Glu197 oxyanion to the center of mass of the hydrogens of the closest water to the phosphorus atom of the inhibited Ser198.

Distance Analysis for Trp82 of the Inhibited Enzyme

To visualize the stability and proximity of the omega loop to the active site of the inhibited enzyme, AMBER's PYTRAJ module was used in order to extract distances as well as an angle. Distance plots were made for the WT and the HID, HIE, and HIP protonation states of the G283H mutation for 2XQJ and 2XQK were made. Important distances from the ε-Nitrogen of the catalytic His438 to the β-carbon (Cβ), the centroid of the amino ring (Cen1), and the centroid of the benzyl ring (Cen2) of Trp82. A summary of the distances and the angle measured are shown below:

Interatomic Distances:

Cβ: Distance from the ε-nitrogen of the catalytic His438 located in the active site to the β-Carbon of Trp82.

Cen1: Distance from the ε-nitrogen of the catalytic His438 located in the active site to the centroid of the amino ring of Trp82.

Cen2: Distance from the ε-nitrogen of the catalytic His438 located in the active site to the centroid of the benzyl ring of Trp82.

Interatomic Angle:

HisNε-Cen1-Cen2: Angle formed by the ε-nitrogen of the catalytic His438 located in the active site to the centroid of the amino ring of Trp82 to the centroid of the benzyl ring of Trp82.

Distance analysis of the acyl loop revealed differences in the positions of Tyr282, Pro285, and Tyr332 for the inhibited WT and the positions of His283, Leu285, and Tyr332 for the inhibited hybrid forms. In the inhibited-WT form of 2XQJ, the hydroxyl group of Tyr282 sits under the acyl-loop and hydrogen bonds to the carbonyl backbone of Val288, thereby restricting the movement of the acyl loop to the active site. However, the interaction of Tyr282 to the backbone of Val288 is not observed in the 2XQK WT inhibited form, suggesting that Tyr282 is much more flexible in the inhibited form of the enzyme. The position of Tyr332 is also much more flexible in the inhibited forms of the enzyme, due to the combination of the Leu285 residue as well as the large isopropoxy group of phosphylated Ser198 “pushing” the α-helix of Tyr332, Phe329, and Glu325. In the HIE forms of 2XQK and 2XQJ, Tyr332 enters the active site once more, but His438 does not climb into the active site because of the strong hydrogen bonding interactions between His438 and Glu197, Glu325, and Glu441. For the HIP and HID forms of 2XQJ there is a significant amount of interaction with the hydroxyl group of Ser79 of the Ω-loop, which correlates with the lack of movement of Trp82. However, in the HIP and HID forms of 2XQK, Tyr332 enters the active site, similar to what happens for the HIE forms of 2XQK and 2XQJ. For the HID and HIP forms of 2XQK and 2XQJ, the His283 mutation sits above the acyl loop near its adjacent residues. For the HIE forms of 2XQK and 2XQJ, the His283 mutation does not sit below the acyl loop, as observed in the HIE form of the native enzyme and lacks any strong hydrogen-bonding interactions even with its adjacent residues.

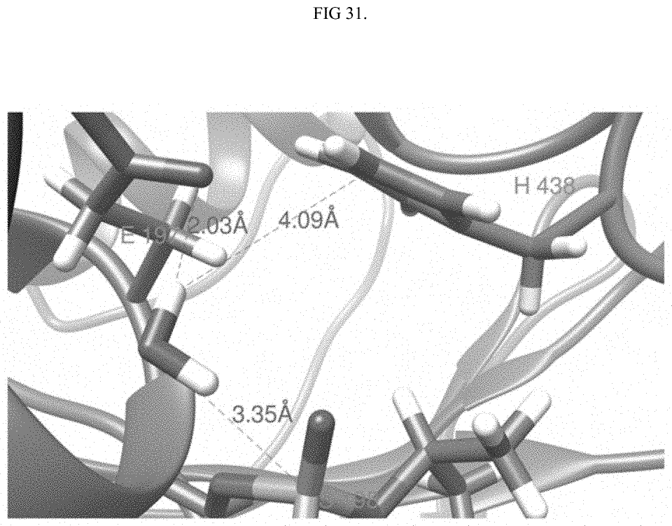

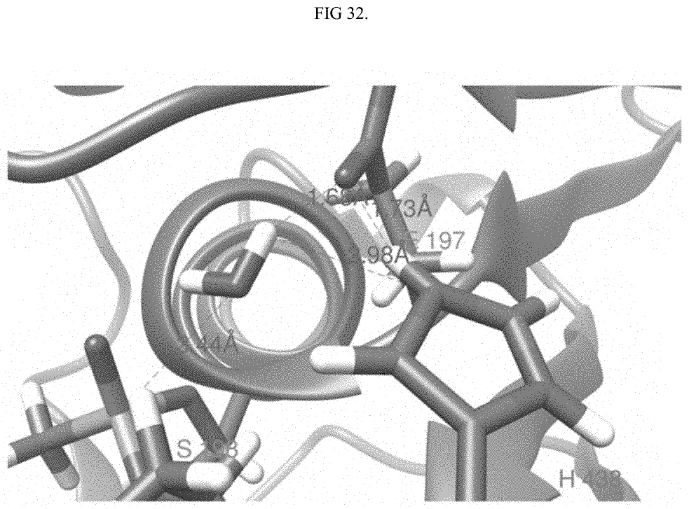

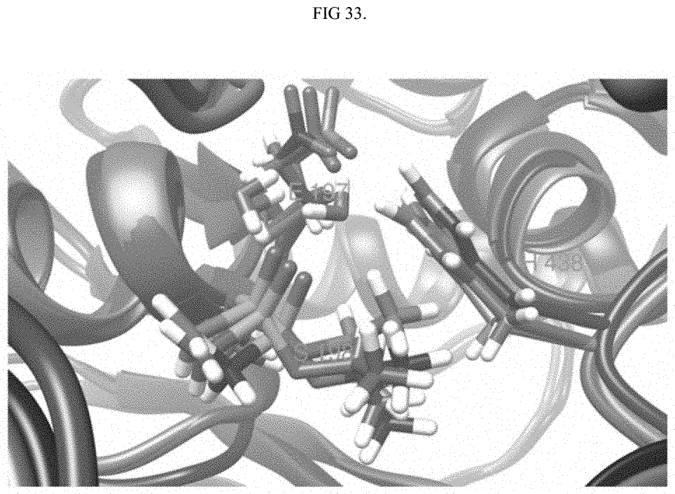

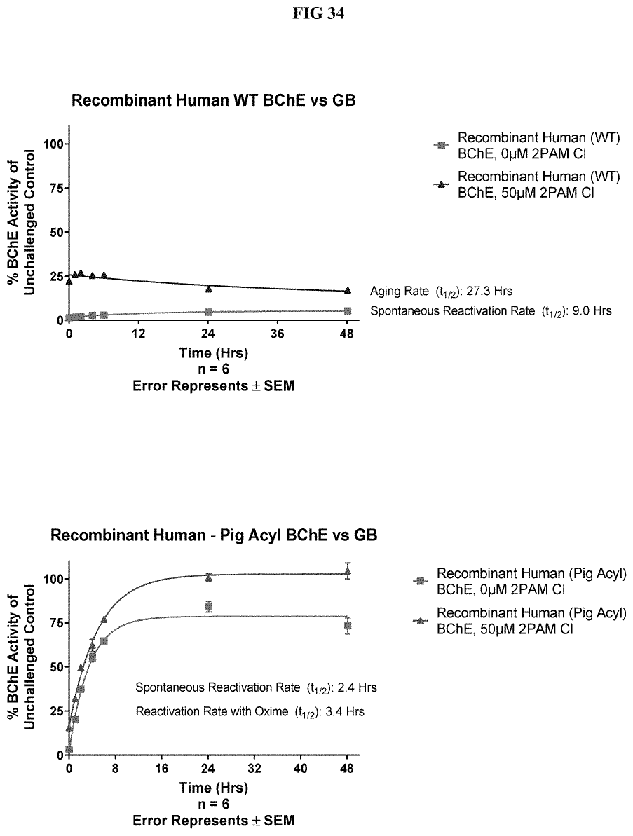

In order to evaluate the reactivation potential of the inhibited WT and hybrid enzyme, two pathways of spontaneous water hydrolysis were evaluated. The two potential pathways are illustrated in Scheme 1, and a number of distances were evaluated to consider either conserved water around the active site residues or potentially catalytic water for reactivation. In pathway #1, it is suggested that if the distance between the oxygen of the closest water to the phosphorus is <4 Å, the distance between the center of mass of the hydrogens of the closest water to HisNε is <4 Å, and the distance between HisHε and the oxyanion of Glu197 is <3 Å, then reactivation is considered possible. In pathway #2, it is suggested that if the distance between the oxygen of the closest water to the phosphorus is <4 Å and the distance between the center of mass of the hydrogens of the closest water to the oxyanion of Glu197 is <4 Å, then this route for reactivation is considered possible. The results of this statistical evaluation of pathways #1 and #2 reveal the generally increased potential for water reactivation in the hybrid forms of the enzyme, as shown in Table 2 where the percentage of frames from the MD trajectory has been counted. This is especially true for the HIE form of the mutation for the 2XQK structure where pathway 1 shows potential for reactivation 19% of the time and for pathway 2 shows potential for reactivation 87% of the time. The potential for reactivation correlates quite well with the distance between the backbone amide hydrogens of Gly115H-Glu197 distance as it creates a small pocket that a water molecule can fit in between the phosphorus of phosphylated Ser198 (as shown in FIGS. 31-33). The reactivity potential can also be amplified by a short distance between HisHε to the oxyanion of Glu197. However, this pathway model is not fully quantitative because the inhibited-WT enzyme of 2XQK shows a larger potential for pathways 1 and 2 than HID.

TABLE 2
Reactivation potential percentages for different species for proposed
spontaneous reactivation pathways 1 and 2 as shown in Scheme 1

| 2XQJ inhibited | Pathway 1 (%) | Pathway 2 (%) |
|---|---|---|
| WT | 0 | 2 |
| HID | 24 | 38 |
| HIE | 0 | 69 |
| HIP | 0 | 62 |

| 2XQK inhibited | Pathway 1 | Pathway 2 |
|---|---|---|
| WT | 15 | 25 |
| HID | 3 | 7 |
| HIE | 19 | 87 |
| HIP | 8 | 51 |

Molecular Dynamics Analysis of the Native Enzyme Before and after Mutation

Molecular dynamics (MD) simulations were carried out for WT and hybrid forms of native BChE, that included each possible protonation states for the His283 in the hybrid form of the enzyme. All MD simulations were performed with the AMBER 16 molecular dynamics package³⁶ with the ff03 force field. The protein preparation was performed for the WT and hybrid forms of the enzyme with 1P0I human native form of BChE (Ref. 42) as previously eluded to in the computational methods.

Clustering Analysis for the Native Enzyme

A clustering protocol using AMBER's CPPTRAJ module was used in order to decrease the amount of structures for analysis in the native forms of the enzyme. A “representative” structure was chosen for each cluster that is color-coded by the relative populations of each cluster.

### DISCUSSION

After observing the unexpected results presented in the corresponding manuscript whereby porcine BChE appears to auto-reactivate following an exposure to sarin, Applicant sought to determine the origin of the differences between the porcine BChE and human BChE by synthesizing a human-porcine hybrid BChE enzyme. It was hypothesized that this hybrid enzyme would be non-immunogenic, allowing for potential use as a therapeutic, while still exhibiting the auto-reactivation capability observed with the porcine BChE. By mutagenizing only four amino acid residues between positions 282 to 285—two within the aminoacyl binding pocket and two adjacent to it—Applicant was able to engineer a hybrid enzyme that appears to possess the capacity to catalytically degrade sarin.

The four amino acid mutations were Y282N, G283H, T284M, and P285L. Although other amino acid differences were observed between the porcine and primate sequences, this stretch of four residues distinctly stood out due to its location in and adjacent to the binding pocket and because it was the only region where consecutive amino acid variations were observed. Two of these residues lie within the aminoacyl binding pocket (positions 284 and 285) and the other two are adjacent to the pocket. Of these four residues, only the human enzyme possesses the rigid proline residue at position 285. All other examined species retain a leucine at position 285. Other research groups have studied various residue mutations within the aminoacyl binding pocket as well, specifically looking at P285, and a mutation to this residue alone does not seem to convey the apparent catalytic activity observed here¹⁶. As such, one might ask what is different about the interactions with the two residues that lie adjacent to the aminoacyl pocket that allow the spontaneous reactivation to occur. Several hypotheses are discussed below.

The simplest explanation for the spontaneous reactivation of the human-porcine BChE hybrid enzyme may be that sarin is not actually covalently bound to the active site serine, but rather sterically occluding the active site. The enzyme then slowly releases the agent, resulting in its natural hydrolysis in the buffer giving the appearance of spontaneous reactivation as the t_{1/2} of both the reactivation and the non-enzymatic hydrolysis of the agent approximate 2 hrs. However, a transient/noncovalent interaction seems unlikely in light of the results of the inhibition and reactivation experiments. Specifically, the IC₅₀ value of the human-porcine hybrid enzyme was approximately 23 nM—closer to that of the human wild type BChE IC₅₀ of 5.8 nM than its porcine counterpart (103 nM). Additionally, the hybrid enzyme clusters with the two wild type enzymes, showing relatively high affinity for the nerve agent (FIG. 4), whereas the mutations that were introduced previously by Lockridge and colleagues impart a much lower affinity for the agent (˜18-45 μM). Furthermore, the fact that 2-PAM Cl appeared to be an effective reactivator of the hybrid enzyme (˜15-30% greater than without 2-PAM Cl; comparable to the ˜20% increase observed with human WT BChE) suggests that sarin is covalently bound since it is understood that the oxime reactivates at the catalytic serine via a nucleophilic attack on the methyl phosphonate group of the phosphylated serine residue. Therefore, these inhibition and reactivation data correlate with the known mechanism of action of the WT enzyme and it seems likely that sarin binds covalently to the active site serine in the hybrid enzyme as well.

The most promising hypothesis is one that is similar to that which has been elucidated with Lockridge's G117H BChE mutation^{20; 49}. It is reasonable to speculate that an RNAse A-like mechanism might be the underlying process for the reactivation that is observed. In the human-porcine hybrid BChE, positions 282-285 were mutagenized from YGTP (SEQ ID NO: 2) to NHML (SEQ ID NO: 4). It is believed that these mutations lead to a greater dynamic capacity of the acyl loop. It is possible that the methionine residue at position 284 perturbs the active site serine such that the histidine at position 283 might activate a water molecule for a nucleophilic attack on the bound OP. One could also speculate that the histidine residue at position 283 alone might serve as the nucleophile which reactivates the catalytic serine. Applicant's modeling data suggest that this distance is likely too great for the histidine to catalyze the reaction on its own accord. However, as discussed previously, the mutations in and adjacent to the acyl pocket could lend to a greater flexibility of the loop, thereby accommodating the reactivation event^{16; 21-24} In particular Leu285 which is the mutated proline residue from the human isoform, facilitates the structural flexibility that is observed and perhaps leads to enhanced reactivation. The Leu285 residue appears to be more dynamic in the porcine-human hybrid. Net, the results presented here open the door to a previously unknown, but promising, “next-generation” bioscavenger.

### EXAMPLE 2 REFERENCES

1. Marrs T C. 1993. Organophosphate poisoning. Pharmacol Ther. 58(1):51-66.
2. Chambers H W. 1992. Organophosphorous compounds: An overview. In: Chambers J E, Levi P E, editors. Organophosphates: Chemistry, fate, and effects. San Diego, Calif. USA: Academic Press, Inc. p. 3-17.
3. Wilson B W, Hooper M J, Hansen M E, Nieberg P S. 1992. Reavtivation of organophosphorous inhibited ache with oximes. In: Chambers J E, Levi P E, editors. Organophosphates: Chemistry, fate, and effects. San Diego, Calif. USA: Academic Press, Inc. p. 107-137.
4. Eto M. 1974. Organophosphorous pesticides: Organic and biological chemistry. Cleveland, Ohio USA: CRC Press.
5. Eddleston M, Buckley N A, Eyer P, Dawson A H. 2008. Management of acute organophosphorus pesticide poisoning. Lancet. 371(9612):597-607.
6. Vellom D C, Radic Z, Li Y, Pickering N A, Camp S, Taylor P. 1993. Amino acid residues controlling acetylcholinesterase and butyrylcholinesterase specificity. Biochemistry. 32(1):12-17.
7. Franjesevic A J, Sillart S B, Beck J M, Vyas S, Callam C S, Hadad C M. 2018. Resurrection and reactivation of acetylcholinesterase and butyrylcholinesterase. Chemistry.
8. Soreq H, Zakut H. 1993. Human cholinesterases and anticholinesterases. San Diego, Calif.: Academic Press, Inc.
9. Nicolet Y, Lockridge O, Masson P, Fontecilla-Camps J C, Nachon F. 2003. Crystal structure of human butyrylcholinesterase and of its complexes with substrate and products. The Journal of biological chemistry. 278(42):41141-41147.
10. Manoharan I, Boopathy R, Darvesh S, Lockridge O. 2007. A medical health report on individuals with silent butyrylcholinesterase in the vysya community of india. Clin Chim Acta. 378(1-2):128-135.
11. Lockridge O. 2015. Review of human butyrylcholinesterase structure, function, genetic variants, history of use in the clinic, and potential therapeutic uses. Pharmacology & therapeutics. 148:34-46.
12. Masson P, Lockridge O. 2010. Butyrylcholinesterase for protection from organophosphorus poisons: Catalytic complexities and hysteretic behavior. Archives of biochemistry and biophysics. 494(2):107-120.
13. Darvesh S, Hopkins D A, Geula C. 2003. Neurobiology of butyrylcholinesterase. Nature reviews Neuroscience. 4(2):131-138.
14. Mumford H, M E P, Lenz D E, Cerasoli D M. 2011. Post-exposure therapy with human butyrylcholinesterase following percutaneous vx challenge in guinea pigs. Clin Toxicol (Phila). 49(4):287-297.
15. Mumford H, Docx C J, Price M E, Green A C, Tattersall J E, Armstrong S J. 2013. Human plasma-derived buche as a stoichiometric bioscavenger for treatment of nerve agent poisoning. Chemico-biological interactions. 203(1):160-166.
16. Lushchekina S V, Schopfer L M, Grigorenko B L, Nemukhin A V, Varfolomeev S D, Lockridge O, Masson P. 2018. Optimization of cholinesterase-based catalytic bioscavengers against organophosphorus agents. Frontiers in pharmacology. 9:211.
17. Nachon F, Brazzolotto X, Trovaslet M, Masson P. 2013. Progress in the development of enzyme-based nerve agent bioscavengers. Chemico-biological interactions. 206(3):536-544.
18. Rice H, Mann T M, Armstrong S J, Price M E, Green A C, Tattersall JEH. 2016. The potential role of bioscavenger in the medical management of nerve-agent poisoned casualties. Chemico-biological interactions. 259(Pt B):175-181.
19. Saxena A, Luo C, Doctor B P. 2008. Developing procedures for the large-scale purification of human serum butyrylcholinesterase. Protein expression and purification. 61(2):191-196.
20. Lockridge O, Blong R M, Masson P, Froment M T, Millard C B, Broomfield C A. 1997. A single amino acid substitution, gly117his, confers phosphotriesterase (organophosphorus acid anhydride hydrolase) activity on human butyrylcholinesterase. Biochemistry. 36(4):786-795.
21. Dafferner A J, Lushchekina S, Masson P, Xiao G, Schopfer L M, Lockridge O. 2017. Characterization of butyrylcholinesterase in bovine serum. Chemico-biological interactions. 266:17-27.
22. Dorandeu F, Foquin A, Briot R, Delacour C, Denis J, Alonso A, Froment M T, Renault F, Lallement G, Masson P. 2008. An unexpected plasma cholinesterase activity rebound after challenge with a high dose of the nerve agent vx. Toxicology. 248(2-3):151-157.
23. Terekhov S S, Smirnov I V, Stepanova A V, Bobik T V, Mokrushina Y A, Ponomarenko N A, Belogurov A A, Jr., Rubtsova M P, Kartseva O V, Gomzikova M O et al. 2017. Microfluidic droplet platform for ultrahigh-throughput single-cell screening of biodiversity. Proceedings of the National Academy of Sciences of the United States of America. 114(10):2550-2555.
24. Biochemical and structural study of a self-reactivating butyrylcholinesterase after v-type nerve agent inhibition. 12th International Meeting of Cholinesterases-6th-Paraoxonase Conference 2015; Elche, Spain.
25. Durocher Y, Perret S, Kamen A. 2002. High-level and high-throughput recombinant protein production by transient transfection of suspension-growing human 293-ebna1 cells. Nucleic acids research. 30(2):E9.
26. Murphy A J M, Kung A L, Swirski R A, Shimke R T. 1992. Cdna expression cloning in human cells using the pλdr2 episomal vector system. Methods. 4(2):111-131.
27. Ellman G L, Courtney K D, Andres V, Jr., Feather-Stone R M. 1961. A new and rapid colorimetric determination of acetylcholinesterase activity. Biochemical pharmacology. 7:88-95.
28. Worek F, Thiermann H, Szinicz L, Eyer P. 2004. Kinetic analysis of interactions between human acetylcholinesterase, structurally different organophosphorus compounds and oximes. Biochemical pharmacology. 68(11):2237-2248.
29. Becke A D. 1993. Density-functional thermochemistry. Iii. The role of exact exchange. The Journal of Chemical Physics. 98(7):5648-5652.
30. Stephens P J, Devlin F J, Chabalowski C F, Frisch M J. 1988. Ab initio calculation of vibrational absorption and circular dichroism spectra using density functional force fields. Journal of Physical Chemistry. 98(45):11623-11627.
31. Kim K, Jordan K D. 1994. Comparison of density functional and mp2 calculations on the water monomer and dimer. Journal of Physical Chemistry. 98(40):10089-10094.
32. Dunning T H. 1989. Gaussian basis sets for use in correlated molecular calculations. I. The atoms boron through neon and hydrogen. The Journal of Chemical Physics. 90(2):1007-1023.
33. Baldridge K, Klamt A. 1997. First principles implementation of solvent effects without outlying charge error. The Journal of Chemical Physics. 106:6622-6633.
34. Cossi M, Barone V, Cammi R, Tomasi J. 1996. Ab initio study of solvated molecules: A new implementation of the polarizable continuum model. Chemical Physics Letters. 255(4-6):327-335.
35. Tomasi J, Mennucci B, Cammi R. 2005. Quantum mechanical continuum solvation models. Chemical Reviews. 105(8):2999-3093.
36. Case D A, Ben-Shalom I Y, Brozell S R, Cerutti D S, Cheatham T E, Cruzeiro III, V. W. D., Darden T A, Duke R E, Ghoreishi D, Gilson M K et al. 2018. Amber 2018.
37. Duan Y, Wu C, Chowdhury S, Lee M C, Xiong G, Zhang W, Yang R, Cieplak P, Luo R, Lee T et al. 2003. A point-charge force field for molecular mechanics simulations of proteins based on condensed-phase quantum mechanical calculations. Journal of Computational Chemistry. 24:1999-2012.
38. Šali A, Blundell T L. 1993. Comparative protein modelling by satisfaction of spatial restraints. Journal of Molecular Biology. 234(3):779-815.
39. Cui Q, Karplus M. 2003. Catalysis and specificity in enzymes: A study of triosephosphate isomerase and comparison with methyl glyoxal synthase. Advances in Protein Chemistry. 66:315-372.
40. Pearlman D A, Case D A, Caldwell J W, Ross W S, Cheatham T E, DeBolt S, Ferguson D, Seibel G, Kollman P. 1995. Amber, a package of computer programs for applying molecular mechanics, normal mode analysis, molecular dynamics and free energy calculations to simulate the structural and energetic properties of molecules. Computer Physics Communications. 91(1-3):1-41.
41. Dolinsky T J, Nielsen J E, McCammon J A, Baker N A. 2004. Pdb2pqr: An automated pipeline for the setup of poisson-boltzmann electrostatics calculations. Nucleic acids research. 32:665-667.
42. Pettersen E F, Goddard T D, Huang C C, Couch G S, Greenblatt D M, Meng E C, Ferrin T E. 2004. Ucsf chimera—a visualization system for exploratory research and analysis. Journal of Computational Chemistry. 25:1605-1612.
43. Wandhammer M, Carletti E, Van Der Schans M, Gillon E, Nicolet Y, Masson P, Goeldner M, Noort D, Nachon F. 2011. Structural study of the complex stereoselectivity of human butyrylcholinesterase for the neurotoxic v-agents. Journal of Biological Chemistry. 286:16783-16789.
44. Shapovalov M V, Dunbrack R L. 2011. A smoothed backbone-dependent rotamer library for proteins derived from adaptive kernel density estimates and regressions. Structure. 19(6):844-858.
45. Jorgensen W L C, J.; Madura, J. D.; Impey, R. W.; Klein, M. L. 1983. Comparison of simple potential functions for simulating liquid water. The Journal of Chemical Physics. 79(2): 926-935.
46. Roe D R, Cheatham T E. 2013. Ptraj and cpptraj: Software for processing and analysis of molecular dynamics trajectory data. Journal of Chemical Theory and Computation. 9:3084-3095.
47. Nachon F, Carletti E, Wandhammer M, Nicolet Y, Schopfer L M, Masson P, Lockridge O. 2011. X-ray crystallographic snapshots of reaction intermediates in the g117h mutant of human butyrylcholinesterase, a nerve agent target engineered into a catalytic bioscavenger. The Biochemical journal. 434(1):73-82.
48. Munro N B, Ambrose K R, Watson A P. 1994. Toxicity of the organophosphate chemical warfare agents ga, gb, and vx: Implications for public protection. Environmental health perspectives. 102(1):18-38.
49. Vyas S, Beck J M, Xia S, Zhang J, Hadad C M. 2010. Butyrylcholinesterase and g116h, g116s, g117h, g117n, e197q and g117h/e197q mutants: A molecular dynamics study. Chemico-biological interactions. 187(1-3):241-245.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A composition comprising a protein having at least 90% sequence identity to wild-type human butyrylcholinesterase, wherein said protein comprises at least one mutation at a position within the acyl binding pocket and at least one mutation adjacent to said acyl biding pocket.

2. A composition comprising a protein having at least 90% sequence identity to wild-type human butyrylcholinesterase, wherein said protein comprises a mutation at a position selected from 282, 283, 284, and combinations thereof.

3. The composition of claim 2, further comprising a mutation at position 285.

4. A composition comprising a protein having at least 90% sequence identity to wild-type human butyrylcholinesterase, wherein said protein comprises a mutation selected from Y282N, G283H, T284M, and combinations thereof.

5. The composition of claim 4, further comprising a P285L mutation.

6. The composition of claim 1, wherein said mutation causes increased catalytic capacity of BChE towards the degradation of cholinesterase inhibitors as compared to wild-type human butyrylcholinesterase.

7. The composition of claim 1, further comprising one or more agents selected from atropine and 2-PAM

8. A method of treating organophosphate exposure in an individual in need thereof, comprising the step of administering a composition according to claim 1.

9. A method of treating organophosphate exposure in an individual in need thereof, comprising the step of administering a composition according to claim 1, wherein said composition is administered to said individual prophylactically.

10. An auto injector device comprising a composition according to claim 1.

11. A method of treating organophosphate exposure in an individual in need thereof, comprising the step of administering said composition to an individual using the autoinjector device of claim 10.
